(19) United States
(12) Patent Application Publication
(10) Pub. No.: US 2025/0369053 A1
(43) Pub. Date: Dec. 4, 2025

(54) RECURRENCE GENE SIGNATURE ACROSS MULTIPLE CANCER TYPES

(71) Applicants: THE HENRY M. JACKSON FOUNDATION FOR THE ADVANCEMENT OF MILITARY MEDICINE, INC., Bethesda, MD (US); WINDBER RESEARCH INSTITUTE, Windber, PA (US); THE UNITED STATES OF AMERICA, AS REPRESENTED BY THE SECRETARY, DEPT OF HEALTH AND HUMAN SERVICES, Bethesda, MD (US)

(72) Inventors: Hai HU, Windber, PA (US); Yi ZHANG, San Diego, CA (US); Albert KOVATICH, Bethesda, MD (US); Maxwell LEE, Bethesda, MD (US); Craig D. SHRIVER, Bethesda, MD (US)

(21) Appl. No.: 19/077,957

(22) Filed: Mar. 12, 2025

Related U.S. Application Data
(62) Division of application No. 17/273,014, filed on Mar. 3, 2021, now abandoned.
(60) Provisional application No. 62/728,339, filed on Sep. 7, 2018.

(51) Int. Cl. C12Q 1/6886; C12Q 1/6809; G01N 33/574

(52) U.S. Cl. CPC C12Q 1/6886; C12Q 1/6809; G01N 33/57484; C12Q 2600/118; C12Q 2600/158

(57) ABSTRACT

The present disclosure provides gene expression profiles that are associated with cancer, including certain gene expression profiles that differentiate between cancer that is at a high risk of recurrence. The gene expression profiles can be measured at the nucleic acid or protein level. The gene expression profiles can also be used to identify a subject for cancer treatment. Also provided are kits for use in predicting cancer recurrence and/or prognosing cancer and an array comprising probes for detecting the unique gene expression profiles associated with cancer.

### CROSS-REFERENCE TO RELATED APPLICATIONS

This application is a Divisional of U.S. application Ser. No. 17/273,014 filed 3 Mar. 2021, which is a U.S. National Stage application of PCT/US2019/049688 filed 5 Sep. 2019, which claims the benefit of, and relies on the filing date of, U.S. provisional patent application No. 62/728,339, filed 7 Sep. 2018, the entire disclosure of which is incorporated herein by reference.

### GOVERNMENT INTEREST

This invention was made with government support under grant number HU0001-16-2-0004, awarded by the Uniformed Services University of the Health Sciences. The government has certain rights in the invention.

### FIELD OF THE INVENTION

The invention relates generally to recurrence gene signatures, and more specifically to recurrence gene signatures for multiple cancer types, such as breast, ovarian, and lung cancers.

### BACKGROUND

Cancer is a leading cause of death worldwide, with the United States having an estimated more than 1,700,000 new cancer diagnoses and over 600,000 cancer fatalities in a single year. Breast cancer is the most common cancer diagnosis in women and the second-leading cause of cancer-related death among women. Major advances in cancer treatment, including breast cancer treatment, over the last 20 years, such as novel chemotherapeutics and other therapies, have led to significant improvement in the rate of survival. Despite the recent advances in cancer treatment, a significant number of patients will still ultimately die from recurrent disease. Thus, there is a need for clinicians to be able to predict the recurrence of a cancer based on the primary cancer of origin, so that treatment decisions can be made accordingly.

The identification of recurrence gene signatures having clinical utility can be used in the management and treatment of cancers. For example, Oncotype Dx® and MammaPrint® are commercially-available PCR and microarray assays that may be used to predict the risk of breast cancer recurrence, based on the expression of specific genes. Both Oncotype Dx® and MammaPrint®, however, which apply to early stage breast cancer cases, are limited to hormonal receptor positive subtypes, with the latter further limited to patients under the age of 61, who have been diagnosed with lymph node-negative breast cancer and have a tumor size less than 5 cm. While gene signatures for other cancer types, such as prostate cancer, are being developed, there exists a need to identify novel gene signature profiles that can be used to predict cancer recurrence across a variety of cancer types.

Therefore, gene signatures that are specific for recurrent cancers that may provide more accurate diagnostic and/or prognostic potential are needed in order to identify individuals who may be susceptible to a recurrence of cancer.

### SUMMARY

Disclosed herein are common gene signatures that may be developed for predicting and prognosing recurrence of various types of cancer, including, for example, breast cancer, such as basal-like subtype breast cancer; ovarian cancer, such as high-grade serous ovarian cancer; and lung cancer, such as squamous cell carcinomas. Gene expression profiles from the gene signatures disclosed herein can be used, for example, to predict the likelihood of a patient developing recurrent cancer, to help understand breast cancer development, or inform treatment decisions. The gene expression profiles can be measured at either the nucleic acid or protein level.

Accordingly, one aspect is directed to gene expression profiles that are associated with multiple cancer types and can be used to predict cancer recurrence in a patient. In this aspect, disclosed herein is a method of obtaining a gene expression profile in a biological sample from a patient, the method comprising detecting expression of a plurality of genes in a biological sample obtained from the patient, wherein the plurality of genes comprises at least 5, such as at least 10, at least 15, at least 20, at least 30, at least 40, at least 50, or at least 60 of the following 63 human genes: PTHLH, LAMB4, P2RX6, OLFM4, CLEC11A, SLC5A5, HSPB1, RPA3, PRMT8, PCDHB5, TRIM67, PGF, PAX1, KLHDC7B, DISP2, LRRC46, P3H4, TM4SF19, SCUBE1, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, IGHV1-3, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, TUNAR, LINC01605, BLOC1S5-TXNDC5, ENSG00000261409, ENSG00000261487, ENSG00000261888, YTHDF3-AS1, ENSG00000271959, ENSG00000272551, ENSG00000272732, and ENSG00000281383 (also referred to herein as the “63-gene signature”). In one embodiment, the gene expression profile comprises all 63 of the aforementioned genes. In certain embodiments, one or more different genes, such as one or more housekeeping genes such as ACTB, GAPDH, HMBS, GUSB, and RPLP0, are used as controls for normalizing expression of the tested genes.

Another aspect is directed to gene expression profiles that are associated with multiple cancer types and can be used to predict cancer recurrence in a patient. In this aspect, disclosed herein is a method of obtaining a gene expression profile in a biological sample from a patient, the method comprising detecting expression of a plurality of genes in a biological sample obtained from the patient, wherein the plurality of genes comprises at least 5, such as at least 10, at least 15, at least 20, at least 30, at least 40, or at least 50 of the following 58 human genes: AGPAT4, BCAS1, SEPT3, GTPBP1, RPA3, CLIP2, GGCX, GRK4, FM05, KCNH3, LRRC46, RNF157, GBGT1, OTOA, ANO10, PPIC, TM2D2, GPR27, GLDC, FAM3B, C6orf120, NRG3, KLK12, UTS2B, RPS3AP47, IGHV1-3, TAX1BP3, ZSWIM7, ENSG00000218073, FAM228B, LINC01615, RPS20P14, FAM225B, CCT8P1, ENSG00000231747, RPS3AP25, KRT8P39, KRT18P5, ENSG00000240211, TCAM1P, ENSG00000240401, ENSG00000243635, PPIAP11, LINC01605, ENSG00000255201, ENSG00000257261, ENSG00000258317, ENSG00000261487, ENSG00000261783, ENSG00000261888, ENSG00000262703, ENSG00000263847, ENSG00000267811, ENSG00000269976, ENSG00000271926, ENSG00000272551, ENSG00000275778, and ENSG00000280241 (also referred to herein as “the 58-gene signature”). In one embodiment, the gene expression profile comprises all 58 of the aforementioned genes. In certain embodiments, one or more different genes, such as one or more housekeeping genes such as ACTB, GAPDH, HMBS, GUSB, and RPLP0, are used as controls for normalizing expression of the tested genes.

In certain embodiments, the plurality of genes comprises at least 2, such as at least 5, at least 10, or 15 of the following 15 genes: RPA3, LRRC46, ANO10, LINC01615, LINC01605, FAM3B, FAM228B, KLK12, IGHV1-3, RPS20P14, ENSG00000231747, ENSG00000240401, ENSG00000261487, ENSG00000261888, and ENSG00000272551 (also referred to herein as “the 15-gene signature”).

In certain embodiments of the method of obtaining a gene expression profile, the biological sample comprises breast cancer, ovarian cancer, or lung cancer. In certain embodiments of the method of obtaining a gene expression profile, the biological sample comprises basal-like subtype breast cancer, high-grade serous ovarian cancer, or squamous cell lung cancer.

These gene expression profiles can be used in a method of collecting data for diagnosing or prognosing recurrent cancer, the method comprising measuring the expression of a representative number of genes in one of the disclosed gene profiles, where gene expression is measured in a sample obtained from a patient. The collected gene expression data can be used to predict whether a subject has recurrent cancer or will develop recurrent cancer and/or to predict severity of the cancer. The collected gene expression data can also be used to inform decisions about treating or monitoring a patient. Given the identification of these unique gene expression profiles, one of skill in the art can determine which of the identified genes to include in the gene profiling analysis. A representative number of genes may include all of the genes listed in a particular profile or some lesser number.

Accordingly, also disclosed herein are methods of predicting cancer recurrence in a cancer patient, the method comprising (1) determining the expression levels of a plurality of genes in a biological sample obtained from the patient, wherein the plurality of genes comprises at least 5, such as at least 10, at least 15, at least 20, at least 30, at least 40, at least 50, or at least 60 of the genes in the 63-gene signature; and (2) determining the risk of cancer recurrence based on reduced or enhanced expression levels of the genes compared to a control sample comprising non-recurrent cancer. In certain embodiments, the method optionally further comprises a step of obtaining from the patient the biological sample. In certain embodiments, the control sample comprising non-recurrent cancer may be a cancer sample from a patient who did not experience cancer recurrence in a given amount of time, such as at least 2 years, at least 5 years, or at least 10 years. In one embodiment, the expression levels of all 63 of the aforementioned genes are determined. In certain embodiments, the cancer patient has basal-like subtype breast cancer, high-grade serous ovarian cancer, or squamous cell lung cancer. In certain embodiments, the high-grade serous ovarian cancer is Stage I, II, or III.

In certain embodiments of the disclosure there is provided a method of predicting cancer recurrence in a cancer patient, the method comprising (1) determining the expression levels of a plurality of genes in a biological sample obtained from a patient, wherein the plurality of genes comprises at least 5, such as at least 10, at least 15, at least 20, at least 30, at least 40, or at least 50 of the genes in the 58-gene signature; and (2) determining the risk of cancer recurrence based on reduced or enhanced expression levels of the genes compared to a control sample. In one embodiment, the expression levels of all 58 of the aforementioned genes are determined. In certain embodiments, the method optionally further comprises a step of obtaining from the patient the biological sample. In certain embodiments, the cancer patient is one who has been previously diagnosed with basal-like subtype breast cancer, high-grade serous ovarian cancer, or squamous cell lung cancer. In certain embodiments, the high-grade serous ovarian cancer is Stage I, II, or III.

In certain embodiments, the expression levels of at least 2, such as at least 5, at least 10, or 15 of the genes in the 15-gene signature are determined.

According to various embodiments, the sample comprises tissue or cells. In certain embodiments, nucleic acid expression is detected, and in yet other embodiments, polypeptide expression is detected.

In various aspects of the method of predicting cancer recurrence in a cancer patient, wherein the expression levels of at least 5, such as at least 10, at least 15, at least 20, at least 30, at least 40, at least 50, or at least 60 of the genes in the 63-gene signature are determined, over-expression of at least one, such as at least 10, at least 15, at least 20, at least 30, at least 40, or at least 50, of the following genes as compared to a control sample or a threshold value indicates a high risk of cancer recurrence in the biological sample: PTHLH, LAMB4, P2RX6, OLFM4, CLEC11A, SLC5A5, HSPB1, RPA3, PRMT8, PCDHB5, TRIM67, PGF, DISP2, LRRC46, P3H4, TM4SF19, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, LINC01605, BLOC1S5-TXNDC5, ENSG00000261487, ENSG00000261888, YTHDF3-AS1, ENSG00000271959, ENSG00000272551, ENSG00000272732, and ENSG00000281383. In various other aspects, under-expression of at least one, such as at least 2 or at least 5, of the following genes as compared to a control sample or a threshold value indicates a high risk of cancer recurrence in the biological sample: PAX1, KLHDC7B, SCUBE1, IGHV1-3, TUNAR, and ENSG00000261409.

In various aspects of the method of predicting cancer recurrence in a cancer patient, wherein the expression levels of at least 5, such as at least 10, at least 15, at least 20, at least 30, at least 40, or at least 50 of the genes in the 58-gene signature are determined, over-expression of at least one, such as at least 10, at least 15, at least 20, least 25, at least 30, or at least 35 of the following genes as compared to a control sample or a threshold value indicates a high risk of cancer recurrence in the biological sample: AGPAT4, BCAS1, RPA3, GGCX, GRK4, FMO5, LRRC46, GBGT1, OTOA, ANO10, PPIC, TM2D2, FAM3B, C6orf120, KLK12, RPS3AP47, TAX1BP3, ZSWIM7, FAM228B, LINC01615, RPS20P14, FAM225B, CCT8P1, ENSG00000231747, RPS3AP25, ENSG00000241211, ENSG00000240401, ENSG00000243635, PPIAP11, LINC01605, ENSG00000257261, ENSG00000261487, ENSG00000261783, ENSG00000261888, ENSG00000267811, ENSG00000269976, ENSG00000271926, ENSG00000272551, and ENSG00000280241. In various other aspects, under-expression of at least one, such as at least 2, at least 5, at least 10, or at least 15 of the following genes as compared to a control sample or a threshold value indicates a high risk of cancer recurrence in the biological sample: SEPT3, GTPBP1, CLIP2, KCNH3, RNF157, GPR27, GLDC, NRG3, UTS2B, IGHV1-3, ENSG00000218073, KRT8P39, KRT18P5, TCAMIP, ENSG00000255201, ENSG00000258317, ENSG00000262703, ENSG00000263847, and ENSG00000275778.

Also disclosed herein is a method of identifying whether a cancer patient, such as basal-like subtype breast cancer patient or a Stage I, II, or III high-grade serous ovarian cancer patient, has a high risk of cancer recurrence, the method comprising (1) determining the expression levels of a plurality of genes in a biological sample from the patient, wherein the plurality of genes comprises at least 5, such as at least 10, at least 15, at least 20, at least 30, at least 40, at least 50, at least 60, or 63 of the genes in the 63-gene signature; (2) determining differential gene expression levels based on reduced or enhanced expression levels of the genes compared to a control non-recurrent cancer sample; (3) calculating a recurrence index for the patient based on the gene expression levels; and (4) identifying the patient as having a high risk of cancer recurrence if the recurrence index is above a threshold. In certain embodiments, the method further comprises calculating the probability of the patient developing cancer recurrence (e.g., within 5 years) based on the recurrence index.

Also disclosed herein is a method of identifying whether a cancer patient, such as basal-like subtype breast cancer patient or a Stage I, II, or III high-grade serous ovarian cancer patient, has a high risk of cancer recurrence, the method comprising (1) determining the expression levels of a plurality of genes in a biological sample from the patient, wherein the plurality of genes comprises at least 5, such as at least 10, at least 15, at least 20, at least 30, at least 40, at least 50, or 58 genes of the 58-gene signature; (2) determining differential gene expression levels based on reduced or enhanced expression levels of the genes compared to a control non-recurrent cancer sample; (3) calculating a recurrence index for the patient based on the gene expression levels; and (4) identifying the patient as having a high risk of cancer recurrence if the recurrence index is above a threshold. In certain embodiments, the method further comprises calculating the probability of the patient developing cancer recurrence (e.g., within 5 years) based on the recurrence index.

In certain embodiments of the methods of identifying whether a cancer patient has a high risk of cancer recurrence disclosed herein, including the method comprising determining the expression levels of a plurality of genes in the 63-gene signature and the method comprising determining the expression levels of a plurality of genes in the 58-gene signature, the patient is identified as having a high risk of recurrence, such as basal-like subtype breast cancer recurrence or Stage I, II, or III high-grade serous ovarian cancer recurrence, if the recurrence index is above a threshold as defined herein.

In certain embodiments of the method comprising determining the expression levels of a plurality of genes in the 63-gene signature, the patient is identified as having a high risk of basal-like subtype breast cancer recurrence if the recurrence index is above a threshold as defined herein. In certain embodiments of the method comprising determining the expression levels of a plurality of genes in the 58-gene signature, the patient is identified as having a high risk of basal-like subtype breast cancer recurrence if the recurrence index is above a threshold as defined herein.

In certain embodiments of the method comprising determining the expression levels of a plurality of genes in the 63-gene signature, the patient is identified as having a high risk of Stage I, II, or III high-grade serous ovarian cancer recurrence if the recurrence index is above a threshold as defined herein, and in certain embodiments of the method comprising determining the expression levels of a plurality of genes in the 58-gene signature, the patient is identified as having a high risk of Stage I, II, or III high-grade serous ovarian cancer recurrence if the recurrence index is above a threshold as defined herein.

Another aspect is directed to kits for use in predicting cancer recurrence and/or prognosing cancer. In one embodiment, the kit comprises a plurality of probes for detecting at least 5, such as at least 10, at least 15, at least 20, at least 30, at least 40, at least 50, or at least 60 of the genes (or polypeptides encoded by the same) of the 63-gene signature. In one embodiment, the kit comprises a plurality of probes for detecting all 63 of the aforementioned genes, and in certain embodiments, the plurality of probes contains probes for detecting no more than 500, no more than 250, no more than 100, or no more than 75 different genes.

In another aspect, there is provided a kit for use in predicting cancer recurrence and/or prognosing cancer, the kit comprising a plurality of probes for detecting at least 5, such as at least 10, at least 15, at least 20, at least 30, at least 40, or at least 50 of the genes (or polypeptides encoded by the same) of the 58-gene signature. In one embodiment, the kit comprises a plurality of probes for detecting all 58 of the aforementioned genes, and in certain embodiments, the plurality of probes contains probes for detecting no more than 500 different genes.

In another aspect, there is provided a kit for use in predicting cancer recurrence and/or prognosing cancer, the kit comprising a plurality of probes for detecting at least 5, such as at least 8, at least 10, or at least 12 of the 15 genes (or polypeptides encoded by the same) of the 15-gene signature. In one embodiment, the kit comprises a plurality of probes for detecting all 15 of the aforementioned genes, and in certain embodiments, the plurality of probes contains probes for detecting no more than 500 different genes.

In certain embodiments, the plurality of probes is selected from a plurality of oligonucleotide probes, a plurality of antibodies, or a plurality of polypeptide probes. In other embodiments, the plurality of probes contains probes for no more than 250, 100, 75, 60, 50, 40, 30, 20, 15, 10, or 5 genes (or polypeptides). In certain embodiments, of the kits disclosed herein, the plurality of probes is attached to the surface of an array, and in certain embodiments, the array comprises no more than 250, 100, 75, 60, 50, 40, 30, 20, 15, 10, or 5 different addressable elements. In one embodiment, the kit further comprises a probe for detecting expression of one or more control genes, and in one embodiment, the plurality of probes is labeled.

The probes on the arrays described herein may be arranged on the substrate within addressable elements to facilitate detection. The array may comprise a limited number of addressable elements so as to distinguish the array from a more comprehensive array, such as a genomic array or the like.

In another aspect, the disclosure provides methods of using the gene expression profiles described herein to identify a patient in need of cancer treatment. The methods can also further comprise a step of treating a patient who has been identified as needing cancer treatment.

### BRIEF DESCRIPTION OF THE DRAWINGS

The accompanying drawings, which are included to provide a further understanding of the disclosure, are incorporated in and constitute a part of this specification. The drawings illustrate embodiments of the disclosure and, together with the detailed description, serve to explain the principles of the disclosure. No attempt is made to show structural details of the disclosure in more detail than may be necessary for a fundamental understanding of the disclosure and various ways in which it may be practiced. A P value of 0 shown in the figures indicates a P value of less than about 0.0001.

FIG. 1A is a Kaplan-Meier plot showing the progression-free interval (PFI) over 10 years for breast cancer patients based on lymph node negative (N0) subtype or lymph node positive (N1, N2, and N3) subtypes.

FIG. 1B is a Kaplan-Meier plot showing the average PFI for breast cancer patients over 10 years based on PAM50 subtype of Luminal A, Luminal B, Her2-enriched, Basal-like, and Normal-like breast cancer.

FIG. 2A is a Kaplan-Meier plot showing the PFI for breast cancer patients over 10 years in the basal-like subtype dataset (n=190) for both patients having a high risk of recurrence and a low risk of recurrence, using a 63-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 2B is a Kaplan-Meier plot showing the disease-free interval (DFI) for breast cancer patients over 10 years in the basal-like subtype dataset (n=190) for both patients having a high risk of recurrence and a low risk of recurrence, using a 63-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 2C is Kaplan-Meier plot showing the overall survival (OS) for breast cancer patients over 10 years in the basal-like subtype dataset (n=190) for both patients having a high risk of recurrence and a low risk of recurrence, using a 63-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 2D is a Kaplan-Meier plot showing the PFI for breast cancer patients over 10 years in the basal-like subtype dataset (n=190) for both patients having a high risk of recurrence and a low risk of recurrence, using a 63-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 2E is a Kaplan-Meier plot showing the DFI for breast cancer patients over 10 years in the basal-like subtype dataset (n=190) for both patients having a high risk of recurrence and a low risk of recurrence, using a 63-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 2F is Kaplan-Meier plot showing the OS for breast cancer patients over 10 years in the basal-like subtype dataset (n=190) for both patients having a high risk of recurrence and a low risk of recurrence, using a 63-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 2G is a Kaplan-Meier plot showing the PFI for breast cancer patients over 10 years in the basal-like subtype dataset (n=190) for both patients having a high risk of recurrence and a low risk of recurrence, using a 63-gene expression signature wherein patients having a recurrence index above the 80^{th} percentile threshold (i.e., those with the highest 20% recurrence index) were categorized as high risk of recurrence and patients having a recurrence index below the 80^{th} percentile threshold were categorized as low risk of recurrence.

FIG. 2H is a Kaplan-Meier plot showing the DFI for breast cancer patients over 10 years in the basal-like subtype dataset (n=190) for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 2I is a Kaplan-Meier plot showing the OS for breast cancer patients over 10 years in the basal-like subtype dataset (n=190) for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 3 is a graph showing the risk of recurrence as a function of a continuous recurrence index score using a 63-gene expression signature and the basal-like subtype dataset (n=190).

FIG. 4A is a Kaplan-Meier plot showing the PFI for breast cancer patients over 10 years in the luminal subtype dataset (n=777) for both patients having a high risk of recurrence and a low risk of recurrence, using a 63-gene expression signature wherein patients having a recurrence index above the 20^{th} percentile threshold were categorized as high risk of recurrence and patients having a recurrence index above the 20^{th} percentile threshold were categorized as low risk of recurrence.

FIG. 4B is a Kaplan-Meier plot showing the DFI for breast cancer patients over 10 years in the luminal subtype dataset (n=777) for both patients having a high risk of recurrence and a low risk of recurrence, using a 63-gene expression signature wherein patients having a recurrence index above the 20^{th} percentile threshold were categorized as high risk of recurrence and patients having a recurrence index above the 20^{th} percentile threshold were categorized as low risk of recurrence.

FIG. 4C is a Kaplan-Meier plot showing the OS for breast cancer patients over 10 years in the luminal subtype dataset (n=777) for both patients having a high risk of recurrence and a low risk of recurrence, using a 63-gene expression signature wherein patients having a recurrence index above the 20^{th} percentile threshold were categorized as high risk of recurrence and patients having a recurrence index above the 20^{th} percentile threshold were categorized as low risk of recurrence.

FIG. 4D is a Kaplan-Meier plot showing the PFI for breast cancer patients over 10 years in the luminal subtype dataset (n=777) for both patients having a high risk of recurrence and a low risk of recurrence, using a 63-gene expression signature wherein patients having a recurrence index above the 50^{th} percentile threshold were categorized as high risk of recurrence and patients having a recurrence index above the 50^{th} percentile threshold were categorized as low risk of recurrence.

FIG. 4E is a Kaplan-Meier plot showing the DFI for breast cancer patients over 10 years in the luminal subtype dataset (n=777) for both patients having a high risk of recurrence and a low risk of recurrence, using a 63-gene expression signature wherein patients having a recurrence index above the 50^{th} percentile threshold were categorized as high risk of recurrence and patients having a recurrence index above the 50^{th} percentile threshold were categorized as low risk of recurrence.

FIG. 4F is a Kaplan-Meier plot showing the OS for breast cancer patients over 10 years in the luminal subtype dataset (n=777) for both patients having a high risk of recurrence and a low risk of recurrence, using a 63-gene expression signature wherein patients having a recurrence index above the 50^{th} percentile threshold were categorized as high risk of recurrence and patients having a recurrence index above the 50^{th} percentile threshold were categorized as low risk of recurrence.

FIG. 4G is a Kaplan-Meier plot showing the PFI for breast cancer patients over 10 years in the luminal subtype dataset (n=777) for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 4H is a Kaplan-Meier plot showing the DFI for breast cancer patients over 10 years in the luminal subtype dataset (n=777) for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 4I is a Kaplan-Meier plot h showing the OS for breast cancer patients over 10 years in the luminal subtype dataset (n=777) for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 5 is a Kaplan-Meier plot showing the PFI for high-grade serous ovarian cancer patients over 15 years based on cancer staging of Stage I, II, III, and IV.

FIG. 6A is a Kaplan-Meier plot showing the PFI for high-grade serous ovarian cancer patients (n=374) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 6B is a Kaplan-Meier plot showing the DFI for high-grade serous ovarian cancer patients (n=374) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 6C is a Kaplan-Meier plot showing the OS for high-grade serous ovarian cancer patients (n=374) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 7A is a Kaplan-Meier plot showing the PFI for Stage I, Stage II, and Stage III high-grade serous ovarian cancer patients (n=314) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 7B is a Kaplan-Meier plot showing the DFI for Stage I, Stage II, and Stage III high-grade serous ovarian cancer patients (n=314) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 7C is a Kaplan-Meier plot showing the OS for Stage I, Stage II, and Stage III high-grade serous ovarian cancer patients (n=314) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 8 is a graph showing the risk of recurrence as a function of a continuous recurrence index score using a 63-gene expression signature and the high-grade serous ovarian cancer subtype dataset (n=374).

FIG. 9A is a Kaplan-Meier plot showing the PFI for Stage IV high-grade serous ovarian cancer patients (n=57) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 9B is a Kaplan-Meier plot showing the OS for Stage IV high-grade serous ovarian cancer patients (n=57) over 10 years for both patients having a high risk of recurrence and a low risk of recurrence, using a 63-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 10A is a Kaplan-Meier plot showing the PFI for breast cancer patients in the basal-like subtype dataset (n=190) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 10B is a Kaplan-Meier plot showing the DFI for breast cancer patients in the basal-like subtype dataset (n=190) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 10C is a Kaplan-Meier plot showing the OS for breast cancer patients in the basal-like subtype dataset (n=190) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 10D is a Kaplan-Meier plot showing the PFI for breast cancer patients in the basal-like subtype dataset (n=190) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 10E is a Kaplan-Meier plot showing the DFI for breast cancer patients in the basal-like subtype dataset (n=190) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 10F is a Kaplan-Meier plot showing the OS for breast cancer patients in the basal-like subtype dataset (n=190) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 10G is a Kaplan-Meier plot showing the PFI for breast cancer patients in the basal-like subtype dataset (n=190) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 10H is a Kaplan-Meier plot showing the DFI for breast cancer patients in the basal-like subtype dataset (n=190) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 10I is a Kaplan-Meier plot showing the OS for breast cancer patients in the basal-like subtype dataset (n=190) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 11 is a graph showing the risk of recurrence as a function of a continuous recurrence index score using a 58-gene expression signature and the basal-like subtype dataset (n=190).

FIG. 12A is a Kaplan-Meier plot showing the PFI for breast cancer patients in the Luminal subtype dataset (n=777) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 12B is a Kaplan-Meier plot showing the DFI for breast cancer patients in the Luminal subtype dataset (n=777) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 12C is a Kaplan-Meier plot showing the OS for breast cancer patients in the Luminal subtype dataset (n=777) over 10 years for both patients having a high risk of recurrence and a low risk of recurrence, using a 58-gene expression signature wherein patients having a recurrence index above the 20^{th} percentile threshold were categorized as high risk of recurrence and patients having a recurrence index below the 20^{th} percentile threshold were categorized as low risk of recurrence.

FIG. 12D is a Kaplan-Meier plot showing the PFI for breast cancer patients in the Luminal subtype dataset (n=777) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 12E is a Kaplan-Meier plot showing the DFI for breast cancer patients in the Luminal subtype dataset (n=777) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 12F is a Kaplan-Meier plot showing the OS for breast cancer patients in the Luminal subtype dataset (n=777) over 10 years for both patients having a high risk of recurrence and a low risk of recurrence, using a 58-gene expression signature wherein patients having a recurrence index above the 50^{th} percentile threshold were categorized as high risk of recurrence and patients having a recurrence index below the 50^{th} percentile threshold were categorized as low risk of recurrence.

FIG. 12G is a Kaplan-Meier plot showing the PFI for breast cancer patients in the Luminal subtype dataset (n=777) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 12H is a Kaplan-Meier plot showing the DFI for breast cancer patients in the Luminal subtype dataset (n=777) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 12I is a Kaplan-Meier plot showing the OS for breast cancer patients in the Luminal subtype dataset (n=777) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 13A is a Kaplan-Meier plot showing the PFI for high-grade serous ovarian cancer patients (n=374) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 13B is a Kaplan-Meier plot showing the DFI for high-grade serous ovarian cancer patients (n=374) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 13C is a Kaplan-Meier plot showing the OS for high-grade serous ovarian cancer patients (n=374) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 14A is a Kaplan-Meier plot showing the PFI for Stage I, Stage II, and Stage III high-grade serous ovarian cancer patients (n=314) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 14B is a Kaplan-Meier plot showing the DFI for Stage I, Stage II, and Stage III high-grade serous ovarian cancer patients (n=314) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 14C is a Kaplan-Meier plot showing the OS for Stage I, Stage II, and Stage III high-grade serous ovarian cancer patients (n=314) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 15 is a graph showing the risk of recurrence as a function of a continuous recurrence index score using a 58-gene expression signature and the high-grade serous ovarian cancer subtype dataset (n=374).

FIG. 16A is a Kaplan-Meier plot showing the PFI for Stage IV high-grade serous ovarian cancer patients (n=57) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

FIG. 16B is a Kaplan-Meier plot showing the OS for Stage IV high-grade serous ovarian cancer patients (n=57) over 10 years for both patients having a high risk of recurrence and a low risk of recurrence, using a 58-gene expression signature wherein patients having a recurrence index above the 80^{th} percentile threshold were categorized as high risk of recurrence and patients having a recurrence index below the 80^{th} percentile threshold were categorized as low risk of recurrence.

The drawings are not necessarily to scale, and may, in part, include exaggerated dimensions for clarity.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

Disclosed herein are methods for diagnosing and prognosing cancer, as well as predicting cancer recurrence across multiple cancer types, including, for example, breast, lung, and ovarian cancer. Both a 63-gene and a 58-gene signature have been developed to predict recurrent disease at or after diagnosis.

### Definitions

In order that the present invention may be more readily understood, certain terms are first defined. Additional definitions are set forth throughout the detailed description.

The term “detecting” or “detection” means any of a variety of methods known in the art for determining the presence or amount of a nucleic acid or a protein. As used throughout the specification, the term “detecting” or “detection” includes either qualitative or quantitative detection.

The term “gene signature” refers to one or more genes or groups of genes having a characteristic pattern of expression that occurs as a result of a pathological condition, such as cancer.

The term “63-gene signature” refers to the following 63 human genes: PTHLH, LAMB4, P2RX6, OLFM4, CLEC11A, SLC5A5, HSPB1, RPA3, PRMT8, PCDHB5, TRIM67, PGF, PAX1, KLHDC7B, DISP2, LRRC46, P3H4, TM4SF19, SCUBE1, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, IGHV1-3, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, TUNAR, LINC01605, BLOC1S5-TXNDC5, ENSG00000261409, ENSG00000261487, ENSG00000261888, YTHDF3-AS1, ENSG00000271959, ENSG00000272551, ENSG00000272732, and ENSG00000281383.

The term “58-gene signature” refers to the following 58 human genes: AGPAT4, BCAS1, SEPT3, GTPBP1, RPA3, CLIP2, GGCX, GRK4, FMO5, KCNH3, LRRC46, RNF157, GBGT1, OTOA, ANO10, PPIC, TM2D2, GPR27, GLDC, FAM3B, C6orf120, NRG3, KLK12, UTS2B, RPS3AP47, IGHV1-3, TAX1BP3, ZSWIM7, ENSG00000218073, FAM228B, LINC01615, RPS20P14, FAM225B, CCT8P1, ENSG00000231747, RPS3AP25, KRT8P39, KRT18P5, ENSG00000240211, TCAM1P, ENSG00000240401, ENSG00000243635, PPIAP11, LINC01605, ENSG00000255201, ENSG00000257261, ENSG00000258317, ENSG00000261487, ENSG00000261783, ENSG00000261888, ENSG00000262703, ENSG00000263847, ENSG00000267811, ENSG00000269976, ENSG00000271926, ENSG00000272551, ENSG00000275778, and ENSG00000280241.

The term “15-gene signature” refers to the following 15 human genes: RPA3, LRRC46, ANO10, LINC01615, LINC01605, FAM3B, FAM228B, KLK12, IGHV1-3, RPS20P14, ENSG00000231747, ENSG00000240401, ENSG00000261487, ENSG00000261888, and ENSG00000272551.

The term “non-recurrent cancer sample” refers to a cancer sample from a patient who did not experience cancer recurrence in a given amount of time after treatment. In certain embodiments, a non-recurrent cancer sample is a cancer sample from a patient who did not experience a cancer recurrence for at least 5 years after treatment.

The term “gene expression profile” refers to the expression levels of a plurality of genes in a sample. As is understood in the art, the expression level of a gene can be analyzed by measuring the expression of a nucleic acid (e.g., genomic DNA or mRNA) or a polypeptide that is encoded by the nucleic acid.

Where available, HUGO Gene Nomenclature Committee (HGNC) annotations are used to describe the genes discussed herein; otherwise, Ensembl gene annotations are used to describe the genes discussed herein. The following Table 1 lists the HGNC annotations, Ensemble gene annotations, Entrezgene numbers, and/or gene name descriptions for the genes discussed herein, where available:

TABLE 1
HGNC and Ensembl Gene Annotations

| HGNC | | Entrezgene | |
|---|---|---|---|
| Symbol | Ensembl Annotation | Number | Description |
| AGPAT4 | ENSG00000026652.13 | 56895 | 1-acylglycerol-3-phosphate O- |
| | | | acyltransferase 4 |
| BCAS1 | ENSG00000064787.13 | 8537 | breast carcinoma amplified sequence 1 |
| SEPT3 | ENSG00000100167.20 | 55964 | septin 3 |
| GTPBP1 | ENSG00000100226.15 | 9567 | GTP binding protein 1 |
| RPA3 | ENSG00000106399.11 | 6119 | replication protein A3 |
| CLIP2 | ENSG00000106665.15 | 7461 | CAP-Gly domain containing linker |
| | | | protein 2 |
| GGCX | ENSG00000115486.11 | 2677 | gamma-glutamyl carboxylase |
| GRK4 | ENSG00000125388.19 | 2868 | G protein-coupled receptor kinase 4 |
| FMO5 | ENSG00000131781.12 | 2330 | flavin containing monooxygenase 5 |
| KCNH3 | ENSG00000135519.6 | 23416 | potassium voltage-gated channel |
| | | | subfamily H member 3 |
| LRRC46 | ENSG00000141294.9 | 90506 | leucine rich repeat containing 46 |
| RNF157 | ENSG00000141576.14 | 114804 | ring finger protein 157 |
| GBGT1 | ENSG00000148288.12 | 26301 | globoside alpha-1,3-N- |
| | | | acetylgalactosaminyltransferase 1 |
| | | | (FORS blood group) |
| OTOA | ENSG00000155719.17 | 146183 | otoancorin |
| ANO10 | ENSG00000160746.12 | 55129 | anoctamin 10 |
| PPIC | ENSG00000168938.5 | 5480 | peptidylprolyl isomerase C |
| TM2D2 | ENSG00000169490.16 | 83877 | TM2 domain containing 2 |
| GPR27 | ENSG00000170837.2 | 2850 | G protein-coupled receptor 27 |
| GLDC | ENSG00000178445.9 | 2731 | glycine decarboxylase |
| FAM3B | ENSG00000183844.16 | 54097 | family with sequence similarity 3 |
| | | | member B |
| C6orf120 | ENSG00000185127.6 | 387263 | chromosome 6 open reading frame |
| | | | 120 |
| NRG3 | ENSG00000185737.12 | 10718 | neuregulin 3 |
| KLK12 | ENSG00000186474.15 | 43849 | kallikrein related peptidase 12 |
| UTS2B | ENSG00000188958.9 | 257313 | urotensin 2B |
| RPS3AP47 | ENSG00000205871.5 | | ribosomal protein S3a pseudogene |
| | | | 47 |
| IGHV1-3 | ENSG00000211935.3 | | immunoglobulin heavy variable 1-3 |
| TAX1BP3 | ENSG00000213977.7 | 30851 | Tax1 binding protein 3 |
| ZSWIM7 | ENSG00000214941.7 | 125150 | zinc finger SWIM-type containing 7 |
| | ENSG00000218073.1 | | |
| FAM228B | ENSG00000219626.8 | 375190 | family with sequence similarity 228 |
| | | | member B |
| LINC01615 | ENSG00000223485.2 | | long intergenic non-protein coding |
| | | | RNA 1615 |
| RPS20P14 | ENSG00000223803.1 | | ribosomal protein S20 pseudogene |
| | | | 14 |
| FAM225B | ENSG00000225684.3 | | family with sequence similarity 225 |
| | | | member B (non-protein coding) |
| CCT8P1 | ENSG00000226015.2 | | chaperonin containing TCP1 subunit |
| | | | 8 pseudogene 1 |
| | ENSG00000231747.1 | | |
| RPS3AP25 | ENSG00000232385.2 | | ribosomal protein S3a pseudogene |
| | | | 25 [Source: HGNC |
| | | | Symbol; Acc: HGNC: 36801] |
| KRT8P39 | ENSG00000233560.2 | | keratin 8 pseudogene 39 |
| KRT18P5 | ENSG00000236670.1 | | keratin 18 pseudogene 5 |
| | ENSG00000240211.1 | | |
| TCAM1P | ENSG00000240280.6 | | testicular cell adhesion molecule 1, |
| | | | pseudogene |
| | ENSG00000240401.8 | | |
| | ENSG00000243635.1 | | |
| PPIAP11 | ENSG00000251495.1 | | peptidylprolyl isomerase A |
| | | | pseudogene 11 |
| LINC01605 | ENSG00000253161.5 | | long intergenic non-protein coding |
| | | | RNA 1605 |
| | ENSG00000255201.1 | | |
| | ENSG00000257261.5 | | |
| | ENSG00000258317.1 | | |
| | ENSG00000261487.1 | | |
| | ENSG00000261783.1 | | |
| | ENSG00000261888.1 | | |
| | ENSG00000262703.1 | | |
| | ENSG00000263847.1 | | |
| | ENSG00000267811.1 | | |
| | ENSG00000269976.1 | | |
| | ENSG00000271926.1 | | |
| | ENSG00000272551.1 | | |
| | ENSG00000275778.2 | | |
| | ENSG00000280241.3 | | |
| PTHLH | ENSG00000087494.15 | 5744 | parathyroid hormone like hormone |
| LAMB4 | ENSG00000091128.12 | 22798 | laminin subunit beta 4 |
| P2RX6 | ENSG00000099957.16 | 9127 | purinergic receptor P2X 6 |
| OLFM4 | ENSG00000102837.6 | 10562 | olfactomedin 4 |
| CLEC11A | ENSG00000105472.12 | 6320 | C-type lectin domain containing 11A |
| SLC5A5 | ENSG00000105641.3 | 6528 | solute carrier family 5 member 5 |
| HSPB1 | ENSG00000106211.8 | 3315 | heat shock protein family B (small) |
| | | | member 1 |
| PRMT8 | ENSG00000111218.11 | 56341 | protein arginine methyltransferase 8 |
| PCDHB5 | ENSG00000113209.8 | 26167 | protocadherin beta 5 |
| TRIM67 | ENSG00000119283.15 | 440730 | tripartite motif containing 67 |
| PGF | ENSG00000119630.13 | 5228 | placental growth factor |
| PAX1 | ENSG00000125813.13 | 5075 | paired box 1 |
| KLHDC7B | ENSG00000130487.6 | 113730 | kelch domain containing 7B |
| DISP2 | ENSG00000140323.5 | 85455 | dispatched RND transporter family |
| | | | member 2 |
| P3H4 | ENSG00000141696.12 | 10609 | prolyl 3-hydroxylase family member |
| | | | 4 (non-enzymatic) |
| TM4SF19 | ENSG00000145107.15 | 116211 | transmembrane 4 L six family |
| | | | member 19 |
| SCUBE1 | ENSG00000159307.18 | 80274 | signal peptide, CUB domain and |
| | | | EGF like domain containing 1 |
| VPS28 | ENSG00000160948.13 | 51160 | VPS28, ESCRT-I subunit |
| SCGB3A1 | ENSG00000161055.3 | 92304 | secretoglobin family 3A member 1 |
| MT2P1 | ENSG00000162840.4 | | metallothionein 2 pseudogene 1 |
| LINC01116 | ENSG00000163364.9 | | long intergenic non-protein coding |
| | | | RNA 1116 |
| CA3 | ENSG00000164879.6 | 761 | carbonic anhydrase 3 |
| OPRPN | ENSG00000171199.10 | 58503 | opiorphin prepropeptide |
| CSN3 | ENSG00000171209.3 | 1448 | casein kappa |
| KCNK3 | ENSG00000171303.6 | 3777 | potassium two pore domain channel |
| | | | subfamily K member 3 |
| GLIS1 | ENSG00000174332.5 | 148979 | GLIS family zinc finger 1 |
| TVP23C | ENSG00000175106.16 | 201158 | trans-golgi network vesicle protein |
| | | | 23 homolog C |
| PCSK1 | ENSG00000175426.10 | 5122 | proprotein convertase |
| | | | subtilisin/kexin type 1 |
| SRRM3 | ENSG00000177679.15 | 222183 | serine/arginine repetitive matrix 3 |
| EXOSC4 | ENSG00000178896.8 | 54512 | exosome component 4 |
| TH | ENSG00000180176.14 | 7054 | tyrosine hydroxylase |
| ZNF703 | ENSG00000183779.6 | 80139 | zinc finger protein 703 |
| MUC12 | ENSG00000205277.9 | 10071 | mucin 12, cell surface associated |
| | ENSG00000213757.3 | | |
| | ENSG00000225840.2 | | |
| TEX41 | ENSG00000226674.9 | | testis expressed 41 (non-protein |
| | | | coding) |
| DNM3OS | ENSG00000230630.4 | | DNM3 opposite strand/antisense |
| | | | RNA |
| LINC00704 | ENSG00000231298.6 | | long intergenic non-protein coding |
| | | | RNA 704 |
| VSIG8 | ENSG00000243284.1 | 391123 | V-set and immunoglobulin domain |
| | | | containing 8 |
| LINC02432 | ENSG00000248810.1 | | long intergenic non-protein coding |
| | | | RNA 2432 |
| | ENSG00000249780.1 | | |
| TUNAR | ENSG00000250366.2 | | TCL1 upstream neural |
| | | | differentiation-associated RNA |
| BLOC1S5- | ENSG00000259040.5 | | BLOC1S5-TXNDC5 readthrough |
| TXNDC5 | | | (NMD candidate) |
| | ENSG00000261409.1 | | |
| YTHDF3- | ENSG00000270673.1 | | YTHDF3 antisense RNA 1 (head to |
| AS1 | | | head) |
| | ENSG00000271959.1 | | |
| | ENSG00000272732.1 | | |
| | ENSG00000281383.1 | | |

The terms “prognosis” and “prognosing” as used herein mean predicting the likelihood of death from the cancer and/or recurrence or metastasis of the cancer within a given time period, with or without consideration of the likelihood that the cancer patient will respond favorably or unfavorably to a chosen therapy or therapies.

As used herein, the term “recurrence index” refers to a numerical index calculated as a weighted linear combination of the expression levels of the genes in a gene signature disclosed herein, such as the 15-, 58-, or 63-gene signatures (or subsets of genes within the gene signatures). In certain embodiments, the weight in the weighted linear combination calculated for each gene represents the importance of a gene's contribution to the prediction of cancer recurrence, and the recurrence index may be calculated as the sum of the weights calculated for each gene. For example, in an embodiment disclosed herein in Example 1 and using the DESeq2 analysis as shown in Table 3, the recurrence index is defined as the summation of the product of the “Base Mean” and the “Stat” for each of the 63 genes.

As used herein, the term “threshold” when used in relation to a recurrence index refers to a numerical value of the recurrence index determined in a representative cohort of cancer patients, such as a representative cohort comprising recurrent and non-recurrent cancer samples or a representative cohort comprising non-recurrent cancer samples, to achieve optimized performance for a gene signature, such as the 15-, 58-, or 63-gene signatures (or subsets of genes within such gene signatures) as disclosed herein. In certain embodiments, the high-risk threshold may be at or above the 50^{th} percentile, such as at or above the top 20^{th} percentile, of the recurrence index values of the representative cohort, wherein the selected threshold may depend on the composition of patients with recurrent cancer in the cohort. In certain embodiments, the low-risk threshold may be below the 50^{th} percentile, such as at or below the bottom 20^{th} percentile, of the recurrence index values of the representative cohort. In another embodiment, the threshold may be determined based on a calculated optimal Receiver Operating Characteristic (ROC) curve.

As used herein, the term “high risk” indicates that a patient has a high likelihood of recurrence or metastasis of the cancer. In certain embodiments, a patient may be considered high risk if the recurrence index calculated for the patient is above a threshold.

The term “isolated,” when used in the context of a polypeptide or nucleic acid refers to a polypeptide or nucleic acid that is substantially free of its natural environment and is thus distinguishable from a polypeptide or nucleic acid that might happen to occur naturally. For instance, an isolated polypeptide or nucleic acid is substantially free of cellular material or other polypeptides or nucleic acids from the cell or tissue source from which it was derived.

The terms “polypeptide,” “peptide,” and “protein” are used interchangeably herein to refer to polymers of amino acids.

The term “polypeptide probe” as used herein refers to a labeled (e.g., isotopically labeled) polypeptide that can be used in a protein detection assay (e.g., mass spectrometry) to quantify a polypeptide of interest in a biological sample.

The term “primer” means a polynucleotide capable of binding to a region of a target nucleic acid, or its complement, and promoting nucleic acid amplification of the target nucleic acid. Generally, a primer will have a free 3′ end that can be extended by a nucleic acid polymerase. Primers also generally include a base sequence capable of hybridizing via complementary base interactions either directly with at least one strand of the target nucleic acid or with a strand that is complementary to the target sequence. A primer may comprise target-specific sequences and optionally other sequences that are non-complementary to the target sequence. These non-complementary sequences may comprise, for example, a promoter sequence or a restriction endonuclease recognition site. One of ordinary skill in the art can design primers to amplify a target sequence that is specific for a target gene of interest.

In the specification, the term “sample” should be understood to mean tumor cells, tumor tissue, non-tumor tissue, conditioned media, blood or blood derivatives (serum, plasma, etc.), urine, or cerebrospinal fluid.

In the specification, the term “recurrence” should be understood to mean the recurrence of the cancer which is being sampled in the patient, in which the cancer has returned to the sampled area after treatment, for example, if sampling breast cancer, recurrence of the breast cancer in the (source) breast tissue. The term should also be understood to mean recurrence of a primary cancer whose site is different to that of the cancer initially sampled, that is, the cancer has returned to a non-sampled area after treatment, such as non-locoregional recurrences. The term “non-recurrent” should be understood to mean the non-recurrence of the cancer which is being sampled in a patient or used as a control, in which the cancer has not returned to the sampled area after treatment and has not returned to a non-sampled area after treatment after a given amount of time, such as 2 years, 5 years, or 10 years after treatment.

### Detecting Gene Expression

As used herein, measuring or detecting the expression of any of the foregoing genes or nucleic acids comprises measuring or detecting any nucleic acid transcript (e.g., mRNA or cDNA) corresponding to the gene of interest or the protein encoded thereby. If a gene is associated with more than one mRNA transcript or isoform, the expression of the gene can be measured or detected by measuring or detecting one or more of the mRNA transcripts of the gene, or all of the mRNA transcripts associated with the gene.

Typically, gene expression can be detected or measured on the basis of mRNA or cDNA levels, although protein levels also can be used when appropriate. Any quantitative or qualitative method for measuring mRNA levels, cDNA, or protein levels can be used. Suitable methods of detecting or measuring mRNA or cDNA levels include, for example, Northern Blotting, microarray analysis, RNA-sequencing, or a nucleic acid amplification procedure, such as reverse-transcription PCR (RT-PCR) or real-time RT-PCR, also known as quantitative RT-PCR (qRT-PCR). Such methods are well known in the art. See e.g., Sambrook et al., Molecular Cloning: A Laboratory Manual, 4^{th} Ed., Cold Spring Harbor Press, Cold Spring Harbor, N.Y., 2012. Other techniques include digital, multiplexed analysis of gene expression, such as the nCounter® (NanoString Technologies, Seattle, WA) gene expression assays, which are further described in US20100112710 and US20100047924.

Detecting a nucleic acid of interest generally involves hybridization between a target (e.g. mRNA or cDNA) and a probe. Sequences of the genes used in various cancer gene expression profiles are known. Therefore, one of skill in the art can readily design hybridization probes for detecting those genes. See, e.g., Sambrook et al., Molecular Cloning: A Laboratory Manual, 4^{th} Ed., Cold Spring Harbor Press, Cold Spring Harbor, N.Y., 2012. For example, polynucleotide probes that specifically bind to the mRNA transcripts of the genes described herein (or cDNA synthesized therefrom) can be created using the nucleic acid sequences of the mRNA or cDNA targets themselves by routine techniques (e.g., PCR or synthesis). As used herein, the term “fragment” means a part or portion of a polynucleotide sequence comprising about 10 or more contiguous nucleotides, about 15 or more contiguous nucleotides, about 20 or more contiguous nucleotides, about 30 or more, or even about 50 or more contiguous nucleotides. In certain embodiments, the polynucleotide probes will comprise 10 or more nucleic acids, 20 or more, 50 or more, or 100 or more nucleic acids. In order to confer sufficient specificity, the probe may have a sequence identity to a complement of the target sequence of about 90% or more, such as about 95% or more (e.g., about 98% or more or about 99% or more) as determined, for example, using the well-known Basic Local Alignment Search Tool (BLAST) algorithm (available through the National Center for Biotechnology Information (NCBI), Bethesda, Md.).

Each probe may be substantially specific for its target, to avoid any cross-hybridization and false positives. An alternative to using specific probes is to use specific reagents when deriving materials from transcripts (e.g., during cDNA production, or using target-specific primers during amplification). In both cases specificity can be achieved by hybridization to portions of the targets that are substantially unique within the group of genes being analyzed, for example hybridization to the polyA tail would not provide specificity. If a target has multiple splice variants, it is possible to design a hybridization reagent that recognizes a region common to each variant and/or to use more than one reagent, each of which may recognize one or more variants.

Stringency of hybridization reactions is readily determinable by one of ordinary skill in the art, and generally is an empirical calculation dependent upon probe length, washing temperature, and salt concentration. In general, longer probes may require higher temperatures for proper annealing, while shorter probes may require lower temperatures. Hybridization generally depends on the ability of denatured nucleic acid sequences to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

“Stringent conditions” or “high stringency conditions,” as defined herein, are identified by, but not limited to, those that: (1) use low ionic strength and high temperature for washing, for example 0.015 M sodium chloride/0.0015 M sodium citrate/0.1% sodium dodecyl sulfate at 50° C.; (2) use during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) use 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash of 0.1×SSC containing EDTA at 55° C. “Moderately stringent conditions” are described by, but not limited to, those in Sambrook et al., Molecular Cloning: A Laboratory Manual, New York: Cold Spring Harbor Press, 1989, and include the use of washing solution and hybridization conditions (e.g., temperature, ionic strength and % SDS) less stringent than those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/mL denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

In certain embodiments, microarray analysis or a PCR-based method is used. In this respect, measuring the expression of the foregoing nucleic acids in a biological sample can comprise, for instance, contacting a sample containing or suspected of containing cancer cells with polynucleotide probes specific to the genes of interest, or with primers designed to amplify a portion of the genes of interest, and detecting binding of the probes to the nucleic acid targets or amplification of the nucleic acids, respectively. Detailed protocols for designing PCR primers are known in the art. See e.g., Sambrook et al., Molecular Cloning: A Laboratory Manual, 4^{th} Ed., Cold Spring Harbor Press, Cold Spring Harbor, N.Y., 2012. In certain embodiments, RNA obtained from a sample may be subjected to qRT-PCR. Reverse transcription may occur by any methods known in the art, such as through the use of an Omniscript RT Kit (Qiagen). The resultant cDNA may then be amplified by any amplification technique known in the art. Gene expression may then be analyzed through the use of, for example, control samples as described below. As described herein, the over- or under-expression of genes relative to controls may be measured to determine a gene expression profile for an individual biological sample. Similarly, detailed protocols for preparing and using microarrays to analyze gene expression are known in the art and described herein.

As used herein, RNA-sequencing (RNA-seq), also called Whole Transcriptome Shotgun Sequencing, refers to any of a variety of high-throughput sequencing techniques used to detect the presence and quantity of RNA transcripts in real time. See Wang, Z., M. Gerstein, and M. Snyder, RNA-Seq: a revolutionary tool for transcriptomics, NAT REV GENET, 2009. 10(1): p. 57-63. RNA-seq can be used to reveal a snapshot of a sample's RNA from a genome at a given moment in time. In certain embodiments, RNA is converted to cDNA fragments via reverse transcription prior to sequencing, and, in certain embodiments, RNA can be directly sequenced from RNA fragments without conversion to cDNA. Adaptors may be attached to the 5′ and/or 3′ ends of the fragments, and the RNA or cDNA may optionally be amplified, for example by PCR. The fragments are then sequenced using high-throughput sequencing technology, such as, for example, those available from Roche (e.g., the 454 platform), Illumina, Inc., and Applied Biosystem (e.g., the SOLID system).

Alternatively or additionally, expression levels of genes can be determined at the protein level, meaning that levels of proteins encoded by the genes discussed herein are measured. Several methods and devices are known for determining levels of proteins including immunoassays, such as described, for example, in U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; 5,458,852; and 5,480,792, each of which is hereby incorporated by reference in its entirety. These assays may include various sandwich, competitive, or non-competitive assay formats, to generate a signal that is related to the presence or amount of a protein of interest. Any suitable immunoassay may be utilized, for example, lateral flow, enzyme-linked immunoassays (ELISA), radioimmunoassays (RIAs), competitive binding assays, and the like. Numerous formats for antibody arrays have been described. Such arrays may include different antibodies having specificity for different proteins intended to be detected. For example, at least 100 different antibodies are used to detect 100 different protein targets, each antibody being specific for one target. Other ligands having specificity for a particular protein target can also be used, such as the synthetic antibodies disclosed in WO 2008/048970, which is hereby incorporated by reference in its entirety. Other compounds with a desired binding specificity can be selected from random libraries of peptides or small molecules. U.S. Pat. No. 5,922,615, which is hereby incorporated by reference in its entirety, describes a device that uses multiple discrete zones of immobilized antibodies on membranes to detect multiple target antigens in an array. Microtiter plates or automation can be used to facilitate detection of large numbers of different proteins.

One type of immunoassay, called nucleic acid detection immunoassay (NADIA), combines the specificity of protein antigen detection by immunoassay with the sensitivity and precision of the polymerase chain reaction (PCR). This amplified DNA-immunoassay approach is similar to that of an enzyme immunoassay, involving antibody binding reactions and intermediate washing steps, except the enzyme label is replaced by a strand of DNA and detected by an amplification reaction using an amplification technique, such as PCR. Exemplary NADIA techniques are described in U.S. Pat. No. 5,665,539 and published U.S. Application 2008/0131883, both of which are hereby incorporated by reference in their entirety. Briefly, NADIA uses a first (reporter) antibody that is specific for the protein of interest and labelled with an assay-specific nucleic acid. The presence of the nucleic acid does not interfere with the binding of the antibody, nor does the antibody interfere with the nucleic acid amplification and detection. Typically, a second (capturing) antibody that is specific for a different epitope on the protein of interest is coated onto a solid phase (e.g., paramagnetic particles). The reporter antibody/nucleic acid conjugate is reacted with sample in a microtiter plate to form a first immune complex with the target antigen. The immune complex is then captured onto the solid phase particles coated with the capture antibody, forming an insoluble sandwich immune complex. The microparticles are washed to remove excess, unbound reporter antibody/nucleic acid conjugate. The bound nucleic acid label is then detected by subjecting the suspended particles to an amplification reaction (e.g. PCR) and monitoring the amplified nucleic acid product.

Although immunoassays have been used for the identification and quantification of proteins, recent advances in mass spectrometry (MS) techniques have led to the development of sensitive, high-throughput MS protein analyses. The MS methods can be used to detect low abundant proteins in complex biological samples. For example, it is possible to perform targeted MS by fractionating the biological sample prior to MS analysis. Common techniques for carrying out such fractionation prior to MS analysis include, for example, two-dimensional electrophoresis, liquid chromatography, and capillary electrophoresis. Selected reaction monitoring (SRM), also known as multiple reaction monitoring (MRM), has also emerged as a useful high-throughput MS-based technique for quantifying targeted proteins in complex biological samples, including prostate cancer biomarkers that are encoded by gene fusions (e.g., TMPRSS2/ERG).

### Samples

The methods described herein involve analysis of gene expression profiles in biological samples obtained from a cancer patient. Cancer cells may be found in a biological sample, such as a tumor, a tissue, or blood. Nucleic acids or polypeptides may be isolated from the sample prior to detecting gene expression. In one embodiment, the biological sample comprises tumor tissue and is obtained through a biopsy. The methods disclosed herein can be used with biological samples collected from a variety of mammals, and in certain embodiments, the methods disclosed herein may be used with biological samples obtained from a human subject.

### Controls

In certain embodiments, the control may be any suitable reference that allows evaluation of the expression level of the genes in the biological sample as compared to the expression of the same genes in a sample comprising control cells. In certain embodiments, the control cells may be non-recurrent cancerous cells, such as cells obtained from a patient or pool of patients who exhibited non-recurrent cancer. Thus, for instance, the control can be a sample that is analyzed simultaneously or sequentially with the test sample, or the control can be the average expression level of the genes of interest in a pool of samples known to be non-recurrent cancer. In certain embodiments, the control is a predetermined “cut-off” or threshold value of absolute expression or calculated recurrence index. Thus, the control can be embodied, for example, in a pre-prepared microarray used as a standard or reference, or in data that reflects the expression profile of relevant genes in a sample or pool of samples known to contain non-recurrent cancer, such as might be part of an electronic database or computer program.

Overexpression and decreased expression (under-expression) of a gene can be determined by any suitable method, such as by comparing the expression of the genes in a test sample with a control gene or threshold value. In certain embodiments, the control gene is one or more housekeeping genes, such as ACTB, GAPDH, HMBS, GUSB, or RPLP0, that can be used to normalize gene expression levels. Regardless of the method used, overexpression and under-expression can be defined as any level of expression greater than or less than the level of expression of a control gene or threshold value. By way of further illustration, overexpression can be defined as expression that is at least about 1.2-fold, 1.5-fold, 2-fold, 2.5-fold, 4-fold, 5-fold, 10-fold, 20-fold, 50-fold, 100-fold higher or even greater expression as compared to tissue control gene or threshold value, and under-expression can similarly be defined as expression that is at least about 1.2-fold, 1.5-fold, 2-fold, 2.5-fold, 4-fold, 5-fold, 10-fold, 20-fold, 50-fold, 100-fold lower or even lower expression as compared to tissue control gene or threshold value.

### Cancer Types and Staging

In various embodiments, the cancer may be selected from testicular, prostate, colorectal, breast, pancreatic, ovarian, cervical, uterine, bone (e.g., osteosarcoma, chondrosarcoma, Ewing's tumor, and chordoma), bladder, skin (e.g., melanoma, squamous cell carcinoma and basal cell carcinoma), blood (e.g., leukemia, lymphoma, and myeloma), lung (e.g., squamous cell carcinoma, adenocarcinoma, large cell carcinoma, small cell carcinoma, and carcinoid tumors), central nervous system, and kidney cancer. In certain embodiments, the cancer is selected from breast cancer, such as basal-like subtype breast cancer; ovarian cancer, such as high-grade serous ovarian cancer; and lung cancer, such as squamous cell carcinoma.

In certain embodiments, the cancer is breast cancer. When diagnosing breast cancer, breast tumors may be classified based on hormone receptor status, such as estrogen receptor (ER), progesterone receptor (PR), and human epidermal growth factor receptor-2 (HER2). Accordingly, the cancer may be characterized as ER+ or ER−, PR+ or PR−, and HER2+ or HER2− (and combinations thereof). Additionally, breast tumors may be classified based on various gene expression features, including luminal A, luminal B, Her2-enriched, basal-like, and normal-like. As known to those of ordinary skill in the art, the basal-like subtype largely overlaps with the “triple negative” subtype (i.e., ER−, PR−, and HER2− based on immunohistochemistry assays of these protein receptors), it being understood that not all basal-like subtype breast cancers are triple negative, and not all triple-negative breast cancers are of the basal-like subtype. As used herein, the basal-like subtype breast cancer mostly, but not exclusively, includes ER−, PR− and HER2−, whereas the luminal subtype is mostly ER+. The breast cancer subtypes may be associated with distinct biological features and clinical prognosis and may be assigned, for example, based on the expression of a panel of 50 genes to predict breast cancer subtypes. See Parker, et al., Supervised Risk Predictor of Breast Cancer Based on Intrinsic Subtype, J. Clin. Oncol. 2009 Mar. 10; 27(8):1160-7.

Many cancers, including breast and ovarian cancers, may be further diagnosed and classified based on the TNM staging system. In the TNM staging system, a tumor stage (T stage), lymph node stage (N stage) and metastases stage (M stage) can be assessed. As used herein, T0 indicates no evidence of tumor; T1 indicates the tumor is less than or equal to 2 cm; T2 indicates the tumor is greater than 2 cm but less than or equal to 5 cm; T3 indicates the tumor is greater than 5 cm; and T4 indicates a tumor of any size growing in the wall of the breast or skin, or inflammatory breast cancer. For lymph node staging, N0 indicates the cancer is not present in any regional lymph nodes; N1 indicates the cancer has spread to 1 to 3 axillary lymph nodes or to one internal mammary lymph node; N2 indicates the cancer has spread to 4 to 9 axillary lymph nodes or to multiple internal mammary lymph nodes; and N3 indicates the cancer has spread to 10 or more axillary lymph nodes, the cancer has spread to the infraclavicular or supraclavicular lymph nodes, the cancer has spread to the internal mammary lymph nodes, or the cancer affects 4 or more axillary lymph nodes and minimum amounts of cancer are in the internal mammary nodes or in sentinel lymph node biopsy. For metastasis staging, M0 indicates there is no spread of the cancer outside of the site of origin, and M1 indicates there is spread to at least one distant organ.

Based on the TNM staging, a cancer may be staged in a range of 0 to IV, wherein stage IV indicates the cancer has metastases; in general, the higher the stage, the poorer the prognosis. Thus, cancers with a high stage (Stage III and Stage IV) have a poorer prognosis for overall survival than cancers with a lower stage (Stage I and Stage II). In general, the lower the stage, the less aggressive the cancer and the better the prognosis (outlook for cure or long-term survival). The higher the stage, the more aggressive the cancer and the poorer the prognosis for long-term, metastases-free survival.

Cancer may also be graded on a scale of G1 to G4, wherein the higher the grade, the more likely the cancer is to grow and spread. G1 indicates that the cells of the biopsied cancerous tissue are well-differentiated, i.e., most like the cells of the tissue of origin (e.g., breast or ovarian tissue), and therefore less likely to spread, and G2 indicates that the cells of the biopsied cancerous tissue are moderately differentiated. G3 and G4 indicate that the cells of the biopsied cancerous tissue are poorly differentiated, and therefore the most likely to spread.

In certain embodiments, the gene expression profiles can be used to prognose cancer, or to predict cancer recurrence, such as basal-like subtype breast cancer recurrence, high-grade serous ovarian cancer recurrence, or squamous cell lung cancer recurrence.

### Arrays

A convenient way of measuring RNA transcript levels for multiple genes in parallel is to use an array (also referred to as microarrays in the art). A useful array may include multiple polynucleotide probes (such as DNA) that are immobilized on a solid substrate (e.g., a glass support such as a microscope slide, or a membrane) in separate locations (e.g., addressable elements) such that detectable hybridization can occur between the probes and the transcripts to indicate the amount of each transcript that is present. The arrays disclosed herein can be used in methods of detecting the expression of a desired combination of genes, which combinations are discussed herein.

In one embodiment, the array comprises (a) a substrate and (b) at least 5, such as at least 10, at least 15, at least 20, at least 30, at least 40, at least 50, at least 60, or 63 different addressable elements that each comprise at least one polynucleotide probe for detecting the expression of an mRNA transcript (or cDNA synthesized from the mRNA transcript) that is specific for one of the genes in the 63-gene signature, such that the array can be used to simultaneously detect the expression of these at least 5, at least 10, at least 15, at least 20, at least 30, at least 40, at least 50, at least 60, or 63 genes.

In one embodiment, the substrate comprises at least 5, such as at least 10, at least 15, at least 20, at least 30, at least 40, at least 50, or 58 different addressable elements, wherein each different addressable element is specific for one of the genes in the 58-gene signature, such that the array can be used to simultaneously detect the expression of these at least at 5, at least 10, at least 15, at least 20, at least 30, at least 40, at least 50, or 58 genes.

In another embodiment, the substrate comprises at least 5, such as at least 10, or 15 different addressable elements, wherein each different addressable element is specific for one of the genes in the 15-gene signature, such that the array can be used to simultaneously detect expression of these at least 5, at least 10, or 15 genes.

In certain embodiments, the array further comprises one or more different addressable elements comprising at least one oligonucleotide probe for detecting the expression of an mRNA transcript (or cDNA synthesized from the mRNA transcript) of a control gene.

As used herein, the term “addressable element” means an element that is attached to the substrate at a predetermined position and specifically binds a known target molecule, such that when target-binding is detected (e.g., by fluorescent labeling), information regarding the identity of the bound molecule is provided on the basis of the location of the element on the substrate. Addressable elements are “different” for the purposes of the present disclosure if they do not bind to the same target gene. The addressable element comprises one or more polynucleotide probes specific for an mRNA transcript of a given gene, or a cDNA synthesized from the mRNA transcript. The addressable element can comprise more than one copy of a polynucleotide or can comprise more than one different polynucleotide, provided that all of the polynucleotides bind the same target molecule. Where a gene is known to express more than one mRNA transcript, the addressable element for the gene can comprise different probes for different transcripts, or probes designed to detect a nucleic acid sequence common to two or more (or all) of the transcripts. Alternatively, the array can comprise an addressable element for the different transcripts. The addressable element also can comprise a detectable label, suitable examples of which are well known in the art.

The array can comprise addressable elements that bind to mRNA or cDNA other than that of the above-reference 63 genes or the above-referenced 58 genes. However, an array capable of detecting a vast number of targets (e.g., mRNA or polypeptide targets), such as arrays designed for comprehensive expression profiling of a cell line, chromosome, genome, or the like, may not be economical or convenient for collecting data to use in diagnosing and/or prognosing cancer. Thus, the array typically comprises no more than about 1000 different addressable elements, such as no more than about 500 different addressable elements, no more than about 250 different addressable elements, or even no more than about 100 different addressable elements, such as about 75 or fewer different addressable elements, about 60 or fewer different addressable elements, about 50 or fewer different addressable elements, about 40 or fewer different addressable elements, about 30 or fewer different addressable elements, about 15 or fewer, about 10 or fewer, or about 5 different addressable elements.

It is also possible to distinguish these diagnostic arrays from the more comprehensive genomic arrays and the like by limiting the number of polynucleotide probes on the array. Thus, in one embodiment, the array has polynucleotide probes for no more than 1000 genes immobilized on the substrate. In other embodiments, the array has oligonucleotide probes for no more than 500, no more than 250, no more than 100, no more than 75, no more than 60, or no more than 50 genes. In certain embodiments, the array has oligonucleotide probes for no more than 40 genes, and in certain embodiments, the array has oligonucleotide probes for no more than 30 genes or no more than 15 genes.

The substrate can be any rigid or semi-rigid support to which polynucleotides can be covalently or non-covalently attached. Suitable substrates include membranes, filters, chips, slides, wafers, fibers, beads, gels, capillaries, plates, polymers, microparticles, and the like. Materials that are suitable for substrates include, for example, nylon, glass, ceramic, plastic, silica, aluminosilicates, borosilicates, metal oxides such as alumina and nickel oxide, various clays, nitrocellulose, and the like.

The polynucleotides of the addressable elements (also referred to as “probes”) can be attached to the substrate in a pre-determined 1- or 2-dimensional arrangement, such that the pattern of hybridization or binding to a probe is easily correlated with the expression of a particular gene. Because the probes are located at specified locations on the substrate (i.e., the elements are “addressable”), the hybridization or binding patterns and intensities create a unique expression profile, which can be interpreted in terms of expression levels of particular genes and can be correlated with prostate cancer in accordance with the methods described herein.

The array can comprise other elements common to polynucleotide arrays. For instance, the array also can include one or more elements that serve as a control, standard, or reference molecule, such as a housekeeping gene or portion thereof, to assist in the normalization of expression levels or the determination of nucleic acid quality and binding characteristics, reagent quality and effectiveness, hybridization success, analysis thresholds and success, etc. These other common aspects of the arrays or the addressable elements, as well as methods for constructing and using arrays, including generating, labeling, and attaching suitable probes to the substrate, consistent with the invention are well-known in the art. Other aspects of the array are as described with respect to the methods disclosed herein.

An array can also be used to measure protein levels of multiple proteins in parallel. Such an array comprises one or more supports bearing a plurality of ligands that specifically bind to a plurality of proteins, wherein the plurality of proteins comprises no more than 500, no more than 250, no more than 100, no more than 75, no more than 60, no more than 50, no more than 40, no more than 30, no more than 15, no more than 10, or no more than 5 different proteins. The ligands are optionally attached to a planar support or beads. In one embodiment, the ligands are antibodies. The proteins that are to be detected using the array correspond to the proteins encoded by the nucleic acids of interest, as described above, including the specific gene expression profiles disclosed. Thus, each ligand (e.g. antibody) is designed to bind to one of the target proteins (e.g., polypeptide sequences encoded by the genes disclosed herein). As with the nucleic acid arrays, each ligand may be associated with a different addressable element to facilitate detection of the different proteins in a sample.

In certain embodiments, disclosed herein are methods of obtaining a gene expression profile in a biological sample, such as a tumor sample, the method comprising: a) incubating an array as disclosed herein with the biological sample; and b) measuring the expression level of the genes of interest.

### Patient Treatment

Disclosed herein are methods of diagnosing, prognosing, and predicting recurrence of cancer in a sample obtained from a sample of a patient, in which gene expression in tumor cells and/or tissues is analyzed. If a sample shows over-expression or under-expression of certain genes relative to a control, for example as represented by the recurrence index, then there is an increased likelihood that the patient's cancer will recur and/or have a worse prognosis than if the sample does not show differential gene expression relative to a control. Thus, the methods of detecting or prognosing cancer may be used to assess the need for therapy or to monitor a response to a therapy (e.g., disease-free recurrence following surgery or other therapy). In the event of such a result, the methods of prognosing cancer may include one or more of the following steps: informing the patient that they are likely to have a cancer recurrence; and treating the patient by an appropriate cancer therapy.

Cancer treatment options include surgery, radiation therapy, hormone therapy, chemotherapy, biological therapy, and/or high intensity focused ultrasound. Drugs approved for cancer are known to the ordinarily skilled artisan based on the cancer type and grade. Thus a method as described herein may, after a positive result, include a further treatment step, such as, surgery, radiation therapy, hormone therapy, chemotherapy, biological therapy, or high intensity focused ultrasound.

Disclosed herein are methods of predicting cancer recurrence in a cancer patient, such as a breast, ovarian, or lung cancer patient, the method comprising (1) testing a biological sample from the patient for the overexpression and/or underexpression of a plurality of genes; (2) calculating a recurrence index for the patient based on the gene overexpression and/or underexpression; and (3) identifying the patient as having a high risk for cancer recurrence if the recurrence index is above a threshold.

In certain embodiments, testing a biological sample from the patient comprises (a) determining the expression levels of a plurality of genes in the biological sample, wherein the plurality of genes comprises at least 5, such as at least 10, at least 15, at least 20, at least 30, at least 40, at least 50, or 57 of the following genes in the 63-gene signature: PTHLH, LAMB4, P2RX6, OLFM4, CLEC11A, SLC5A5, HSPB1, RPA3, PRMT8, PCDHB5, TRIM67, PGF, DISP2, LRRC46, P3H4, TM4SF19, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, LINC01605, BLOC1S5-TXNDC5, ENSG00000261487, ENSG00000261888, YTHDF3-AS1, ENSG00000271959, ENSG00000272551, ENSG00000272732, and ENSG00000281383; and (b) determining differential gene expression based on enhanced expression levels of the plurality of genes compared to a control non-recurrent cancer sample.

In certain embodiments, testing a biological sample from the patient comprises (a) determining the expression levels of a plurality of genes in the biological sample, wherein the plurality of genes comprises at least 2, such as at least 3, at least 4, at least 5, or 6 of the following genes in the 63-gene signature: PAX1, KLHDC7B, SCUBE1, IGHV1-3, TUNAR, and ENSG00000261409; and (b) determining differential gene expression based on reduced expression levels of the plurality of genes compared to a control non-recurrent cancer sample.

In certain embodiments, testing a biological sample from the patient comprises (a) determining the expression levels of a plurality of genes in the biological sample, wherein the plurality of genes comprises at least 5, such as at least 10, at least 15, at least 20, at least 30, or 39 of the following genes in the 58-gene signature: AGPAT4, BCAS1, RPA3, GGCX, GRK4, FMO5, LRRC46, GBGT1, OTOA, ANO10, PPIC, TM2D2, FAM3B, C6orf120, KLK12, RPS3AP47, TAX1BP3, ZSWIM7, FAM228B, LINC01615, RPS20P14, FAM225B, CCT8P1, ENSG00000231747, RPS3AP25, ENSG00000241211, ENSG00000240401, ENSG00000243635, PPIAP11, LINC01605, ENSG00000257261, ENSG00000261487, ENSG00000261783, ENSG00000261888, ENSG00000267811, ENSG00000269976, ENSG00000271926, ENSG00000272551, and ENSG00000280241; and (b) determining differential gene expression based on enhanced expression levels of the plurality of genes compared to a control non-recurrent cancer sample.

In certain embodiments, testing a biological sample from the patient comprises (a) determining the expression levels of a plurality of genes in the biological sample, wherein the plurality of genes comprises at least 2, such as at least 5, at least 6, at least 7, at least 8, at least 9, at least 10, at least 15, or 19 of the following genes in the 58-gene signature: SEPT3, GTPBP1, CLIP2, KCNH3, RNF157, GPR27, GLDC, NRG3, UTS2B, IGHV1-3, ENSG00000218073, KRT8P39, KRT18P5, TCAMIP, ENSG00000255201, ENSG00000258317, ENSG00000262703, ENSG00000263847, and ENSG00000275778; and (b) determining differential gene expression based on reduced expression levels of the plurality of genes compared to a control non-recurrent cancer sample

In certain embodiments, the plurality of genes comprises at least 5, such as at least 10, at least 15, such as at least 20, at least 30, at least 40, at least 50, at least 60, or 63 of the genes in the 63-gene signature. In certain embodiments, the plurality of genes comprises at least 5, at least 10, at least 15, at least 20, at least 30, at least 40, at least 50, or 58 of the genes in the 58-gene signature. In other embodiments, the plurality of genes comprises at least 2, at least 5, or at least 10 of the genes in the 15-gene signature.

In certain embodiments of the disclosure, a patient may be identified as having a high risk of cancer recurrence by determining differential gene expression levels based on reduced or enhanced expression levels of genes compared to a control non-recurrent cancer sample, and identifying the patient as having a high risk of cancer recurrence if the recurrence index calculated based on gene expression levels is above a threshold. In certain embodiments, the cancer is basal-like subtype breast cancer, and in the certain embodiments, the cancer is Stage I, II, or III high-grade serous ovarian cancer.

### Kits

The polynucleotide probes and/or primers or antibodies or polypeptide probes that can be used in the methods described herein can be arranged in a kit. Thus, one embodiment is directed to a kit for diagnosing, prognosing, or predicting the recurrence of cancer comprising a plurality of polynucleotide probes for detecting at least 5, such as at least 10, at least 15, at least 20, at least 30, at least 40, at least 50, or at least 60 of the genes in the 63-gene signature, wherein the plurality of polynucleotide probes contains polynucleotide probes for no more than 500, 250, 100, 75, 60, 50, 40, 30, 20, 15, 10, or 5 genes. In one embodiment, the plurality of polynucleotide probes comprises polynucleotide probes for detecting all 63 of the aforementioned genes.

Another embodiment is directed to a kit for diagnosing, prognosing, or predicting the recurrence of cancer comprising a plurality of polynucleotide probes for detecting at least 5, at least 10, at least 15, at least 20, at least 30, at least 40, or at least 50 of the genes in the 58-gene signature, wherein the plurality of polynucleotide probes contains polynucleotide probes for no more than 500, 250, 100, 75, 60, 50, 40, 30, 20, 15, 10, or 5 genes. In one embodiment, the plurality of polynucleotide probes comprises polynucleotide probes for detecting all 58 of the aforementioned genes.

In yet another embodiment, there is provided a kit for diagnosing, prognosing, or predicting the recurrence of cancer comprising a plurality of polynucleotide probes for detecting at least 2, at least 5, or at least 10, or 15 of the genes in the 15-gene signature, wherein the plurality of polynucleotide probes contains polynucleotide probes for no more than 500, 250, 100, 75, 60, 50, 40, 30, 20, 15, 10, or 5 genes.

In one embodiment, the kit comprises at least one oligonucleotide probe for detecting the expression of a control gene. The polynucleotide probes may be optionally labeled.

The kit may optionally include polynucleotide primers for amplifying a portion of the mRNA transcripts from at least 5, at least 10, at least 15, at least 20, at least 30, at least 40, at least 50, or at least 60 of the genes in the 63-gene signature. In one embodiment, the kit optionally includes polynucleotide primers for amplifying a portion of the mRNA transcripts from all 63 of the aforementioned genes.

In one embodiment, the kit optionally includes polynucleotide primers for amplifying a portion of the mRNA transcripts from at least 5, at least 10, at least 15, at least 20, at least 30, at least 40, or at least 50 of the genes in the 58-gene signature. In one embodiment, the kit optionally includes polynucleotide primers for amplifying a portion of the mRNA transcripts from the all 58 of the aforementioned genes. In one embodiment, the kit comprises polynucleotide primers for amplifying a portion of the mRNA transcripts from a control gene.

In another embodiment, the kit optionally includes polynucleotide primers for amplifying a portion of the mRNA transcripts from at least 2, at least 5, at least 10, or 15 of the genes in the 15-gene signature.

The kit for diagnosing, prognosing, or predicting recurrence of cancer may also comprise antibodies. Thus, in one embodiment, the kit for diagnosing, prognosing, or predicting recurrence of cancer comprises a plurality of antibodies for detecting at least 5, at least 10, at least 15, at least 20, at least 30, at least 40, at least 50, at least 60, or 63 of the polypeptides encoded by genes in the 63-gene signature, wherein the plurality of antibodies contains antibodies for no more than 500, 250, 100, 75, 60, 50, 40, 30, 20, 15, 10, or 5 polypeptides.

In one embodiment, the kit for diagnosing, prognosing, or predicting recurrence of cancer comprises a plurality of antibodies for detecting at least 5, at least 10, at least 15, at least 20, at least 30, at least 40, at least 50, or 58 of the polypeptides encoded by the genes in the 58-gene signature, wherein the plurality of antibodies contains antibodies for no more than 500, 250, 100, 75, 60, 50, 40, 30, 20, 15, 10, or 5 polypeptides.

In another embodiment, the kit for diagnosing, prognosing, or predicting recurrence of cancer comprises a plurality of antibodies for detecting at least 2, at least 5, at least 10, or 15 the genes in the 15-gene signature, wherein the plurality of antibodies contains antibodies for no more than 500, 250, 100, 75, 60, 50, 40, 30, 20, 15, 10, or 5 polypeptides. The antibodies may be optionally labeled.

As noted above, the polynucleotide or polypeptide probes and antibodies described herein may be optionally labeled with a detectable label. Any detectable label used in conjunction with probe or antibody technology, as known by one of ordinary skill in the art, can be used. As described herein, the labelled polynucleotide probes or labelled antibodies are not naturally occurring molecules; that is the combination of the polynucleotide probe coupled to the label or the antibody coupled to the label do not exist in nature. In certain embodiments, the probe or antibody is labeled with a detectable label selected from the group consisting of a fluorescent label, a chemiluminescent label, a quencher, a radioactive label, biotin, mass tags and/or gold.

In one embodiment, a kit includes instructional materials disclosing methods of use of the kit contents in a disclosed method. The instructional materials may be provided in any number of forms, including, but not limited to, written form (e.g., hardcopy paper, etc.), in an electronic form (e.g., computer diskette or compact disk) or may be visual (e.g., video files). The kits may also include additional components to facilitate the particular application for which the kit is designed. Thus, for example, the kits may additionally include other reagents routinely used for the practice of a particular method, including, but not limited to buffers, enzymes, labeling compounds, and the like. Such kits and appropriate contents are well known to those of skill in the art. The kit can also include a reference or control sample. The reference or control sample can be a biological sample or a data base.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

Unless indicated otherwise in these Examples, the methods involving commercial kits were done following the instructions of the manufacturers.

In the examples that follow, gene signatures for breast cancer recurrence was developed using RNA-seq data. The initial signature was then validated using other public datasets as well as an internal dataset.

### Example 1

In 2006, The Cancer Genome Atlas (TCGA) was established to coordinate an effort to comprehensively characterize molecular events in primary cancers and to provide these data to the public. By the end of the project, TCGA had characterized the molecular landscape of tumors from 11,160 patients across 33 cancer types and defined their many molecular subtypes. The TCGA data, available through Bioconductor's TCGAbiolinks package, makes it possible to compare and contrast multiple cancer types in order to identify common themes that transcend the tissue of origin. With the completion of the TCGA project across 33 different cancer types, the largest ever set of molecular data from six experimental platforms, including RNA-Seq and whole-exome sequencing, is publicly available.

The TCGAbiolinks package was used to download breast cancer RNA-Seq data. Raw count data from the harmonized database were downloaded, interrogating 56,963 annotated genes of 1,222 samples. 1,102 samples were from primary tumors; 7 samples from recurrent tumors and 113 samples from normal tissues were excluded from the analysis. Clinical data were provided by Windber Research Institute for 1,097 patients. Taken together, 1,090 patients had both RNA-Seq data and clinical data available, and thus were used in the analyses described herein. The sequencing depth ranged from 13 million to 114 million, with a median of 58 million. Table 2 below details the clinical data for the 1,090 samples used in the analyses that follow.

TABLE 2
Breast Cancer Patient Clinical Characteristics

| | | RNA-seq (N = 1090) | |
|---|---|---|---|
| Factors | | N (%) | |
| Age | Median (min, max) | 58 | (26, 90) |
| Gender | Female | 1078 | (99%) |
| | Male | 12 | (1%) |
| Menopausal Status | Pre-menopausal | 226 | (21%) |
| | Peri-menopausal | 39 | (4%) |
| | Post-menopausal | 702 | (64%) |
| | Indeterminate | 34 | (3%) |
| | Unknown | 89 | (8%) |
| Race | White | 752 | (69%) |
| | Black | 182 | (17%) |
| | Asian | 61 | (5%) |
| | Indian | 1 | (0%) |
| | Unknown | 94 | (9%) |
| Tumor (T) Stage | T1 | 279 | (26%) |
| | T2 | 631 | (58%) |
| | T3 | 137 | (12%) |
| | T4 | 40 | (4%) |
| | Unknown | 3 | (0%) |
| Node (N) Stage | N0 | 514 | (47%) |
| | N1 | 360 | (33%) |
| | N2 | 120 | (11%) |
| | N3 | 76 | (7%) |
| | Unknown | 20 | (2%) |
| Metastasis (M) | M0 | 907 | (83%) |
| Stage | M1 | 22 | (2%) |
| | Unknown | 161 | (15%) |
| Estrogen Receptor | Positive | 803 | (74%) |
| (ER) | Negative | 237 | (22%) |
| | Unknown | 50 | (4%) |
| Progesterone | Positive | 694 | (64%) |
| Receptor (PR) | Negative | 343 | (31%) |
| | Unknown | 53 | (5%) |
| Her2/neu (Her2) | Positive | 168 | (15%) |
| Status | Negative | 895 | (82%) |
| | Unknown | 27 | (3%) |
| PAM50 Cluster | Lum A | 563 | (52%) |
| | Lum B | 215 | (20%) |
| | Her2-enriched | 82 | (7%) |
| | Basal-like | 190 | (17%) |
| | Normal | 40 | (4%) |
| Overall Survival | Death | 151 | (14%) |
| | Alive | 939 | (86%) |
| Disease-Free | Event | 84 | (8%) |
| Interval (DFI) | Event-free | 864 | (79%) |
| | Unknown | 142 | (13%) |
| Progression-Free | Event | 145 | (13%) |
| Interval (PFI) | Event-free | 945 | (87%) |

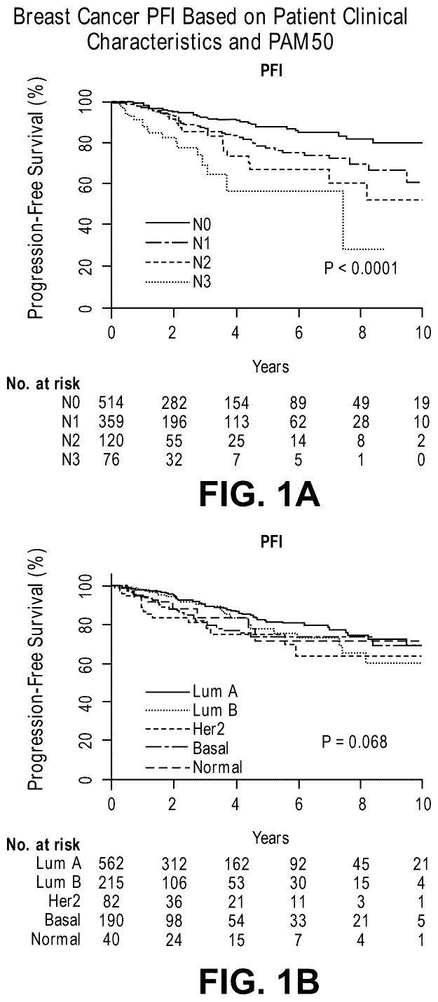

FIG. 1A is a Kaplan-Meier plot showing breast cancer PFI over a 10-year period based on lymph-node staging N0-N1, and FIG. 1B is a Kaplan-Meier plot showing breast cancer PFI over a 10-year period based on molecular subtype.

For the analysis, only basal-like subtype cases of Stages I, II, and III (N=190) were analyzed. Those having progression events within 2 years (N=18) were compared to those having no progression events for at least 5 years (N=40). Table A below details the clinical data for the 190 samples used in the analyses that follow.

TABLE A
Basal-like Subtype Breast Cancer Patient Clinical Characteristics

| | RSA-seq (N = 190) |
|---|---|
| Factors | N (%) |

| | | | |
|---|---|---|---|
| Age | Median (min, max) | 54 | (26, 90) |
| Menopausal Status | Pre-menopausal | 38 | (22%) |
| | Peri-menopausal | 10 | (6%) |
| | Post-menopausal | 117 | (66%) |
| | Indeterminate | 11 | (6%) |
| | Unknown | 14 | (7%) |
| Race | White | 111 | (58%) |
| | Black | 64 | (34%) |
| | Asian | 7 | (4%) |
| | Unknown | 8 | (4%) |
| Tumor (T) Stage | T1 | 37 | (19%) |
| | T2 | 127 | (67%) |
| | T3 | 19 | (10%) |
| | T4 | 6 | (3%) |
| | Unknown | 1 | (1%) |
| Node (N) Stage | N0 | 118 | (62%) |
| | N1 | 51 | (27%) |
| | N2 | 15 | (8%) |
| | N3 | 6 | (3%) |
| Metastasis (M) | M0 | 165 | (87%) |
| Stage | M1 | 4 | (2%) |
| | Unknown | 21 | (11%) |
| Estrogen Receptor | Positive | 21 | (11%) |
| (ER) | Negative | 162 | (85%) |
| | Unknown | 7 | (4%) |
| Progesterone | Positive | 12 | (6%) |
| Receptor (PR) | Negative | 169 | (90%) |
| | Unknown | 9 | (5%) |
| Her2/neu (Her2) | Positive | 6 | (3%) |
| Status | Negative | 180 | (95%) |
| | Unknown | 4 | (2%) |
| Disease-Free | Event | 22 | (12%) |
| Events | | | |
| Progression-Free | Event | 29 | (15%) |
| Events | | | |

Three RNA-Seq analysis methods were evaluated: (1) DESeq2; (2) edgeR; and (3) voom/limma. DESeq2 analysis uses negative binomial generalized linear models with gene-specific dispersion parameters, tested by either Wald test or likelihood ratio test (LRT). EdgeR analysis uses negative binomial generalized linear models with both common and gene-specific dispersion parameters moderated by empirical Bayes to borrow information across genes, tested by LRT or quasi-likelihood F-test. Voom/limma analysis does not assume negative binomial distributions, instead estimating the mean-variance relationship of the log-counts, generating a precision weight for each normalized observation, which are entered into the normal distribution-based limma empirical Bayes analysis pipeline or any other microarray analysis methods.

31,375 genes (56% of all genes) had less than or equal to 10 counts in 90% of the samples, not providing meaningful analysis. Thus, they were excluded from further analysis. As a result, 25,228 genes were retained for further analysis.

For TMM Normalization, Log counts per million (CPM) were measured for both raw data and TMM normalized data.

DESeq2 Analysis: 3,296 genes (13%) had a p value less than 0.05. Using Benjamini & Hochberg false discovery rate (FDR) adjustment, 307 genes remained to be significant (adjusted p value <0.05).

edgeR Analysis: 3,296 genes (14%) had a p value less than 0.05. Using Benjamini & Hochberg FDR adjustment, 343 genes remained to be significant (adjusted p value <0.01).

Voom/limma Analysis: 1,152 genes (4.6%) had a p value less than 0.05. Using Benjamini & Hochberg FDR adjustment, no genes remained to be significant (adjusted p value <0.05). 228 genes had a p value less than 0.01.

A total of 63 genes were identified as differentially expressed by both DESeq2 and edgeR, as shown in Tables 3 and 4, respectively. A total of 58 genes were identified as differentially expressed by both DESeq2 and voom/limma, as shown below in Tables 5 and 6, respectively. There were 15 genes that overlapped both the 63-gene signature and the 58-gene signature.

TABLE 3
Gene Expression from DESeq2 Analysis for 63-Gene Signature

| | HGNC | | | | | |
|---|---|---|---|---|---|---|
| | Symbol or | | Log2 | | | |
| | Ensembl | Base | Fold | | | p-value |
| | annotation | Mean | Change | Stat | p-value | adjusted |
| 1 | PTHLH | 453.05 | 2.63 | 5.58 | 2.47E−08 | 4.45E−05 |
| 2 | LAMB4 | 20.73 | 1.47 | 3.74 | 0.0002 | 0.0252 |
| 3 | P2RX6 | 28.21 | 2.98 | 5.73 | 1.03E−08 | 3.25E−05 |
| 4 | OLFM4 | 2655.02 | 3.97 | 5.67 | 1.44E−08 | 3.30E−05 |
| 5 | CLEC11A | 714.71 | 1.85 | 4.93 | 8.05E−07 | 0.0006 |
| 6 | SLC5A5 | 41.65 | 2.65 | 5.12 | 3.09E−07 | 0.0003 |
| 7 | HSPB1 | 21764.85 | 1.67 | 4.21 | 2.51E−05 | 0.0072 |
| 8 | RPA3 | 1370.60 | 0.79 | 4.76 | 1.90E−06 | 0.0012 |
| 9 | PRMT8 | 4.18 | 2.73 | 3.79 | 0.0001 | 0.0215 |
| 10 | PCDHB5 | 97.07 | 2.28 | 5.62 | 1.90E−08 | 4.00E−05 |
| 11 | TRIM67 | 35.80 | 2.69 | 5.23 | 1.74E−07 | 0.0002 |
| 12 | PGF | 884.27 | 1.75 | 5.46 | 4.75E−08 | 7.48E−05 |
| 13 | PAX1 | 91.64 | −3.45 | −4.87 | 1.12E−06 | 0.0008 |
| 14 | KLHDC7B | 4250.29 | −3.08 | −5.56 | 2.65E−08 | 4.46E−05 |
| 15 | DISP2 | 219.15 | 1.73 | 4.16 | 3.21E−05 | 0.0084 |
| 16 | LRRC46 | 61.65 | 0.99 | 4.31 | 1.63E−05 | 0.0051 |
| 17 | P3H4 | 2197.19 | 1.29 | 4.14 | 3.44E−05 | 0.0088 |
| 18 | TM4SF19 | 33.43 | 1.66 | 3.65 | 0.0003 | 0.0314 |
| 19 | SCUBE1 | 173.39 | −2.55 | −5.26 | 1.48E−07 | 0.0002 |
| 20 | ANO10 | 2745.48 | 0.77 | 4.09 | 4.24E−05 | 0.0100 |
| 21 | VPS28 | 8819.73 | 1.08 | 4.12 | 3.77E−05 | 0.0092 |
| 22 | SCGB3A1 | 118.90 | 2.92 | 4.61 | 3.95E−06 | 0.0019 |
| 23 | MT2P1 | 13.07 | 1.83 | 4.12 | 3.74E−05 | 0.0091 |
| 24 | LINC01116 | 159.68 | 1.60 | 4.75 | 2.03E−06 | 0.0012 |
| 25 | CA3 | 296.84 | 2.32 | 4.57 | 4.78E−06 | 0.0022 |
| 26 | OPRPN | 1072.49 | 8.35 | 6.78 | 1.23E−11 | 3.10E−07 |
| 27 | CSN3 | 1685.65 | 6.53 | 6.02 | 1.76E−09 | 8.89E−06 |
| 28 | KCNK3 | 434.72 | 2.37 | 4.48 | 7.33E−06 | 0.0030 |
| 29 | GLIS1 | 84.23 | 2.70 | 5.99 | 2.16E−09 | 9.08E−06 |
| 30 | TVP23C | 221.18 | 1.33 | 4.68 | 2.89E−06 | 0.0016 |
| 31 | PCSK1 | 122.85 | 1.67 | 3.72 | 0.0002 | 0.0261 |
| 32 | SRRM3 | 147.28 | 2.34 | 5.30 | 1.15E−07 | 0.0002 |
| 33 | EXOSC4 | 2696.70 | 1.24 | 4.16 | 3.12E−05 | 0.0083 |
| 34 | TH | 24.06 | 2.60 | 4.24 | 2.22E−05 | 0.0066 |
| 35 | ZNF703 | 2019.85 | 1.40 | 4.37 | 1.22E−05 | 0.0043 |
| 36 | FAM3B | 207.09 | 2.72 | 5.59 | 2.22E−08 | 4.30E−05 |
| 37 | KLK12 | 53.75 | 3.09 | 4.01 | 6.16E−05 | 0.0130 |
| 38 | MUC12 | 30.25 | 1.98 | 4.37 | 1.24E−05 | 0.0043 |
| 39 | IGHV1-3 | 112.02 | −3.31 | −5.38 | 7.38E−08 | 0.0001 |
| 40 | ENSG00000 | 120.07 | 1.83 | 4.55 | 5.48E−06 | 0.0024 |
| | 213757 | | | | | |
| 41 | FAM228B | 364.07 | 0.85 | 4.66 | 3.13E−06 | 0.0016 |
| 42 | LINC01615 | 89.64 | 1.83 | 4.92 | 8.54E−07 | 0.0006 |
| 43 | RPS20P14 | 85.39 | 1.62 | 5.01 | 5.32E−07 | 0.0004 |
| 44 | ENSG00000 | 37.45 | 3.13 | 4.70 | 2.61E−06 | 0.0015 |
| | 225840 | | | | | |
| 45 | TEX41 | 59.45 | 2.31 | 6.21 | 5.46E−10 | 4.59E−06 |
| 46 | DNM3OS | 299.34 | 2.11 | 4.55 | 5.40E−06 | 0.0024 |
| 47 | LINC00704 | 27.30 | 2.72 | 4.27 | 1.95E−05 | 0.0060 |
| 48 | ENSG00000 | 100.41 | 1.77 | 5.05 | 4.51E−07 | 0.0004 |
| | 231747 | | | | | |
| 49 | ENSG00000 | 36.09 | 0.92 | 3.44 | 0.0006 | 0.0492 |
| | 240401 | | | | | |
| 50 | VSIG8 | 24.56 | 1.84 | 4.57 | 4.89E−06 | 0.0022 |
| 51 | LINC02432 | 30.58 | 2.35 | 3.50 | 0.0005 | 0.0433 |
| 52 | ENSG00000 | 9.98 | 1.85 | 3.85 | 0.0001 | 0.0196 |
| | 249780 | | | | | |
| 53 | TUNAR | 273.72 | −6.02 | −5.67 | 1.42E−08 | 3.30E−05 |
| 54 | LINC01605 | 31.35 | 1.30 | 3.58 | 0.0003 | 0.0376 |
| 55 | BLOC1S5- | 36.82 | 2.03 | 4.52 | 6.18E−06 | 0.0026 |
| | TXNDC5 | | | | | |
| 56 | ENSG00000 | 32.08 | −4.53 | −4.76 | 1.96E−06 | 0.0012 |
| | 261409 | | | | | |
| 57 | ENSG00000 | 11.15 | 1.11 | 4.32 | 1.58E−05 | 0.0050 |
| | 261487 | | | | | |
| 58 | ENSG00000 | 55.46 | 1.56 | 4.80 | 1.56E−06 | 0.0010 |
| | 261888 | | | | | |
| 59 | YTHDF3- | 112.04 | 1.73 | 4.62 | 3.77E−06 | 0.0018 |
| | AS1 | | | | | |
| 60 | ENSG00000 | 20.30 | 1.43 | 4.25 | 2.14E−05 | 0.0064 |
| | 271959 | | | | | |
| 61 | ENSG00000 | 6.62 | 2.01 | 4.48 | 7.42E−06 | 0.0030 |
| | 272551 | | | | | |
| 62 | ENSG00000 | 26.95 | 1.43 | 3.86 | 0.0001 | 0.0195 |
| | 272732 | | | | | |
| 63 | ENSG00000 | 54.52 | 1.98 | 4.73 | 2.24E−06 | 0.0013 |
| | 281383 | | | | | |

TABLE 4
Gene Expression from edgeR Analysis for 63-Gene Signature

| | HGNC | | | | | |
|---|---|---|---|---|---|---|
| | symbol or | | | | | |
| | Ensembl | | | | | |
| | annotation | LogFC | LogCPM | F | p-value | FDR |
| 1 | PTHLH | 3.62 | 3.82 | 47.22 | 3.97E−09 | 5.27E−06 |
| 2 | LAMB4 | 4.43 | 0.88 | 60.50 | 1.11E−10 | 6.69E−07 |
| 3 | P2RX6 | 2.92 | −0.92 | 31.80 | 4.76E−07 | 0.0002 |
| 4 | OLFM4 | 5.35 | 8.47 | 28.36 | 1.56E−06 | 0.0004 |
| 5 | CLEC11A | 1.79 | 3.66 | 23.33 | 9.67E−06 | 0.0015 |
| 6 | SLC5A5 | 2.63 | −0.35 | 25.12 | 4.98E−06 | 0.0009 |
| 7 | HSPB1 | 1.64 | 8.60 | 17.12 | 0.0001 | 0.0087 |
| 8 | RPA3 | 0.77 | 4.63 | 22.26 | 1.45E−05 | 0.0021 |
| 9 | PRMT8 | 4.23 | −0.54 | 24.45 | 6.37E−06 | 0.0011 |
| 10 | PCDHB5 | 2.24 | 0.81 | 30.02 | 8.72E−07 | 0.0003 |
| 11 | TRIM67 | 4.37 | 1.27 | 42.74 | 1.47E−08 | 1.33E−05 |
| 12 | PGF | 1.69 | 3.97 | 29.70 | 9.76E−07 | 0.0003 |
| 13 | PAX1 | −4.88 | 2.11 | 18.22 | 7.04E−05 | 0.0065 |
| 14 | KLHDC7B | −3.08 | 6.26 | 17.76 | 8.48E−05 | 0.0073 |
| 15 | DISP2 | 1.73 | 1.99 | 16.84 | 0.0001 | 0.0093 |
| 16 | LRRC46 | 0.93 | 0.19 | 16.63 | 0.0001 | 0.0099 |
| 17 | P3H4 | 1.25 | 5.29 | 17.07 | 0.0001 | 0.0088 |
| 18 | TM4SF19 | 2.70 | 0.38 | 23.30 | 9.75E−06 | 0.0015 |
| 19 | SCUBE1 | −2.58 | 1.68 | 18.12 | 7.32E−05 | 0.0066 |
| 20 | ANO10 | 0.74 | 5.63 | 16.76 | 0.0001 | 0.0095 |
| 21 | VPS28 | 1.04 | 7.31 | 16.87 | 0.0001 | 0.0092 |
| 22 | SCGB3A1 | 4.16 | 3.04 | 26.33 | 3.20E−06 | 0.0007 |
| 23 | MT2P1 | 2.22 | −1.27 | 18.19 | 7.13E−05 | 0.0066 |
| 24 | LINC01116 | 1.54 | 1.52 | 21.43 | 1.99E−05 | 0.0026 |
| 25 | CA3 | 3.32 | 3.20 | 34.75 | 1.79E−07 | 8.85E−05 |
| 26 | OPRPN | 9.78 | 7.17 | 56.88 | 2.81E−10 | 1.01E−06 |
| 27 | CSN3 | 7.77 | 8.57 | 33.57 | 2.64E−07 | 0.0001 |
| 28 | KCNK3 | 2.34 | 2.96 | 18.31 | 6.78E−05 | 0.0064 |
| 29 | GLIS1 | 2.64 | 0.59 | 33.46 | 2.74E−07 | 0.0001 |
| 30 | TVP23C | 1.16 | 1.93 | 18.87 | 5.43E−05 | 0.0054 |
| 31 | PCSK1 | 2.90 | 2.47 | 24.59 | 6.05E−06 | 0.0011 |
| 32 | SRRM3 | 2.26 | 1.38 | 25.89 | 3.76E−06 | 0.0008 |
| 33 | EXOSC4 | 1.21 | 5.59 | 17.41 | 9.77E−05 | 0.0078 |
| 34 | TH | 3.88 | 0.75 | 26.29 | 3.25E−06 | 0.0007 |
| 35 | ZNF703 | 1.35 | 5.17 | 18.36 | 6.65E−05 | 0.0063 |
| 36 | FAM3B | 2.70 | 1.90 | 29.19 | 1.16E−06 | 0.0003 |
| 37 | KLK12 | 3.06 | −0.02 | 17.67 | 8.79E−05 | 0.0074 |
| 38 | MUC12 | 1.89 | −0.84 | 17.55 | 9.25E−05 | 0.0076 |
| 39 | IGHV1-3 | −4.21 | 1.88 | 20.23 | 3.16E−05 | 0.0038 |
| 40 | ENSG0000 | 1.78 | 1.11 | 19.51 | 4.21E−05 | 0.0045 |
| | 0213757 | | | | | |
| 41 | FAM228B | 0.80 | 2.70 | 19.67 | 3.95E−05 | 0.0043 |
| 42 | LINC01615 | 1.78 | 0.70 | 22.91 | 1.13E−05 | 0.0017 |
| 43 | RPS20P14 | 1.60 | 0.66 | 24.36 | 6.57E−06 | 0.0011 |
| 44 | ENSG0000 | 3.87 | 1.10 | 24.10 | 7.23E−06 | 0.0012 |
| | 0225840 | | | | | |
| 45 | TEX41 | 2.21 | 0.08 | 36.69 | 9.57E−08 | 5.89E−05 |
| 46 | DNM3OS | 2.00 | 2.38 | 18.17 | 7.17E−05 | 0.0066 |
| 47 | LINC00704 | 2.66 | −0.96 | 18.29 | 6.83E−05 | 0.0064 |
| 48 | ENSG0000 | 1.73 | 0.86 | 24.56 | 6.12E−06 | 0.0011 |
| | 0231747 | | | | | |
| 49 | ENSG0000 | 1.47 | −0.28 | 23.43 | 9.29E−06 | 0.0015 |
| | 0240401 | | | | | |
| 50 | VSIG8 | 1.79 | −1.10 | 19.96 | 3.53E−05 | 0.0040 |
| 51 | LINC02432 | 3.70 | 1.31 | 19.93 | 3.56E−05 | 0.0040 |
| 52 | ENSG0000 | 3.45 | −0.72 | 31.78 | 4.80E−07 | 0.0002 |
| | 0249780 | | | | | |
| 53 | TUNAR | −6.00 | 2.34 | 19.01 | 5.13E−05 | 0.0053 |
| 54 | LINC01605 | 3.28 | 0.56 | 46.34 | 5.11E−09 | 5.86E−06 |
| 55 | BLOC1S5- | 1.87 | −0.62 | 17.68 | 8.75E−05 | 0.0074 |
| | TXNDC5 | | | | | |
| 56 | ENSG0000 | −6.47 | 2.41 | 17.68 | 8.76E−05 | 0.0074 |
| | 0261409 | | | | | |
| 57 | ENSG0000 | 1.07 | −2.10 | 17.88 | 8.09E−05 | 0.0071 |
| | 0261487 | | | | | |
| 58 | ENSG0000 | 1.52 | 0.04 | 21.93 | 1.64E−05 | 0.0023 |
| | 0261888 | | | | | |
| 59 | YTHDF3- | 1.68 | 1.01 | 20.10 | 3.33E−05 | 0.0039 |
| | AS1 | | | | | |
| 60 | ENSG0000 | 1.39 | −1.33 | 17.48 | 9.49E−05 | 0.0077 |
| | 0271959 | | | | | |
| 61 | ENSG0000 | 1.88 | −2.79 | 18.96 | 5.22E−05 | 0.0053 |
| | 0272551 | | | | | |
| 62 | ENSG0000 | 2.07 | −0.58 | 27.45 | 2.15E−06 | 0.0005 |
| | 0272732 | | | | | |
| 63 | ENSG0000 | 1.92 | −0.01 | 21.10 | 2.25E−05 | 0.0029 |
| | 0281383 | | | | | |

TABLE 5
Gene Expression from DESeq2 Analysis for 58-Gene Signature

| | HGNC | | | | | |
|---|---|---|---|---|---|---|
| | Symbol or | | Log₂ | | | |
| | Ensembl | Base | Fold | | | p-value |
| | annotation | Mean | Change | Stat | p-value | adjusted |
| 1 | AGPAT4 | 1062.86 | 0.98 | 3.58 | 0.0003 | 0.0374 |
| 2 | BCAS1 | 83.67 | 1.56 | 3.83 | 0.0001 | 0.0199 |
| 3 | SEPT3 | 3255.57 | −1.55 | −4.02 | 5.93E−05 | 0.0128 |
| 4 | GTPBP1 | 3929.05 | −0.50 | −4.03 | 5.69E−05 | 0.0127 |
| 5 | RPA3 | 1370.6 | 0.79 | 4.76 | 1.90E−06 | 0.0012 |
| 6 | CLIP2 | 1742.09 | −0.92 | −4.43 | 9.59E−06 | 0.0036 |
| 7 | GGCX | 3338.34 | 0.47 | 3.54 | 0.000399 | 0.0407 |
| 8 | GRK4 | 206.98 | 0.65 | 4.20 | 2.72E−05 | 0.0075 |
| 9 | FMO5 | 267.61 | 1.27 | 3.69 | 0.0002 | 0.0285 |
| 10 | KCNH3 | 52.75 | −1.32 | −3.98 | 7.00E−05 | 0.0139 |
| 11 | LRRC46 | 61.65 | 0.96 | 4.31 | 1.63E−05 | 0.0051 |
| 12 | RNF157 | 226.66 | −1.37 | −3.70 | 0.0002 | 0.0274 |
| 13 | GBGT1 | 683.69 | 1.03 | 3.56 | 0.0004 | 0.0388 |
| 14 | OTOA | 19.97 | 1.29 | 4.09 | 4.32E−05 | 0.0101 |
| 15 | ANO10 | 2745.48 | 0.77 | 4.09 | 4.24E−05 | 0.0100 |
| 16 | PPIC | 3046.29 | 0.78 | 3.66 | 0.000252 | 0.0308 |
| 17 | TM2D2 | 3164.17 | 0.92 | 4.03 | 5.66E−05 | 0.0127 |
| 18 | GPR27 | 553.69 | −1.40 | −3.63 | 0.0003 | 0.0333 |
| 19 | GLDC | 815.44 | −2.36 | −4.45 | 8.53E−06 | 0.0033 |
| 20 | FAM3B | 207.09 | 2.72 | 5.59 | 2.22E−08 | 4.30E−05 |
| 21 | C6orf120 | 1216.28 | 0.58 | 3.43 | 0.0006 | 0.0497 |
| 22 | NRG3 | 26.95 | −2.47 | −5.12 | 2.98E−07 | 0.0003 |
| 23 | KLK12 | 53.75 | 3.09 | 4.01 | 6.16E−05 | 0.0130 |
| 24 | UTS2B | 16.11 | −1.30 | −3.43 | 0.0006 | 0.0496 |
| 25 | RPS3AP47 | 38.44 | 1.08 | 3.94 | 8.06E−05 | 0.0152 |
| 26 | IGHV1-3 | 112.02 | −3.31 | −5.38 | 7.38E−08 | 0.0001 |
| 27 | TAX1BP3 | 1982.64 | 0.66 | 3.87 | 0.0001 | 0.0188 |
| 28 | ZSWIM7 | 959.66 | 0.64 | 3.48 | 0.0005 | 0.0452 |
| 29 | ENSG00000 | 6.40 | −1.67 | −3.84 | 0.0001 | 0.0196 |
| | 218073 | | | | | |
| 30 | FAM228B | 364.07 | 0.85 | 4.66 | 3.13E−06 | 0.0016 |
| 31 | LINC01615 | 89.64 | 1.83 | 4.92 | 8.54E−07 | 0.0006 |
| 32 | RPS20P14 | 85.39 | 1.62 | 5.01 | 5.32E−07 | 0.0004 |
| 33 | FAM225B | 54.74 | 1.39 | 4.18 | 2.90E−05 | 0.0079 |
| 34 | CCT8P1 | 59.44 | 0.89 | 4.19 | 2.75E−05 | 0.0075 |
| 35 | ENSG00000 | 100.41 | 1.77 | 5.05 | 4.51E−07 | 0.0004 |
| | 231747 | | | | | |
| 36 | RPS3AP25 | 7.46 | 1.21 | 3.48 | 0.0005 | 0.0456 |
| 37 | KRT8P39 | 10.17 | −1.02 | −3.58 | 0.0003 | 0.0372 |
| 38 | KRT18P5 | 18.94 | −1.17 | −3.90 | 9.44E−05 | 0.0169 |
| 39 | ENSG00000 | 9.29 | 1.05 | 3.51 | 0.0004 | 0.0431 |
| | 240211 | | | | | |
| 40 | TCAM1P | 198.96 | −2.52 | −4.59 | 4.37E−06 | 0.0020 |
| 41 | ENSG00000 | 36.09 | 0.92 | 3.44 | 0.0006 | 0.0492 |
| | 240401 | | | | | |
| 42 | ENSG00000 | 2.24 | 1.71 | 3.50 | 0.0005 | 0.0435 |
| | 243635 | | | | | |
| 43 | PPIAP11 | 23.41 | 0.88 | 3.50 | 0.0005 | 0.0437 |
| 44 | LINC01605 | 31.35 | 1.30 | 3.58 | 0.0003 | 0.0377 |
| 45 | ENSG00000 | 34.88 | −2.44 | −4.64 | 3.56E−06 | 0.0018 |
| | 255201 | | | | | |
| 46 | ENSG00000 | 38.89 | 0.88 | 4.01 | 6.18E−05 | 0.0130 |
| | 257261 | | | | | |
| 47 | ENSG00000 | 7.51 | −1.07 | −3.46 | 0.0005 | 0.0471 |
| | 258317 | | | | | |
| 48 | ENSG00000 | 11.15 | 1.11 | 4.32 | 1.58E−05 | 0.0050 |
| | 261487 | | | | | |
| 49 | ENSG00000 | 16.12 | 1.25 | 3.86 | 0.000116 | 0.0195 |
| | 261783 | | | | | |
| 50 | ENSG00000 | 55.46 | 1.56 | 4.80 | 1.56E−06 | 0.0010 |
| | 261888 | | | | | |
| 51 | ENSG00000 | 10.68 | −1.20 | −3.50 | 0.0005 | 0.0435 |
| | 262703 | | | | | |
| 52 | ENSG00000 | 9.30 | −1.12 | −3.81 | 0.0001 | 0.0208 |
| | 263847 | | | | | |
| 53 | ENSG00000 | 6.42 | 1.33 | 3.79 | 0.0001 | 0.0215 |
| | 267811 | | | | | |
| 54 | ENSG00000 | 7.75 | 1.38 | 3.90 | 9.80E−05 | 0.0174 |
| | 269976 | | | | | |
| 55 | ENSG00000 | 17.63 | 0.97 | 3.66 | 0.0002 | 0.0308 |
| | 271926 | | | | | |
| 56 | ENSG00000 | 6.62 | 2.01 | 4.48 | 7.42E−06 | 0.0030 |
| | 272551 | | | | | |
| 57 | ENSG00000 | 7.87 | −1.36 | −3.59 | 0.0003 | 0.0365 |
| | 275778 | | | | | |
| 58 | ENSG00000 | 27.57 | 1.89 | 3.94 | 8.29E−05 | 0.0154 |
| | 280241 | | | | | |

TABLE 6
Gene Expression from Voom/Limma
Analysis for 58-Gene Signature

| | HGNC | | | | | |
|---|---|---|---|---|---|---|
| | Symbol or | | | | | |
| | Ensemble | Ave. | | | Adjusted | |
| | annotation | Expression | t | p-value | p-value | B |
| 1 | AGPAT4 | 3.85 | 3.05 | 0.0034 | 0.9832 | −3.89 |
| 2 | BCAS1 | −0.39 | 3.50 | 0.001194 | 0.9832 | −4.13 |
| 3 | SEPT3 | 5.00 | −4.45 | 3.75E−05 | 0.6209 | −3.20 |
| 4 | GTPBP1 | 6.07 | −4.20 | 8.83E−05 | 0.6209 | −2.98 |
| 5 | RPA3 | 4.47 | 4.05 | 0.0001 | 0.7294 | −3.26 |
| 6 | CLIP2 | 4.79 | −3.07 | 0.0032 | 0.9832 | −3.84 |
| 7 | GGCX | 5.82 | 2.72 | 0.0084 | 0.9832 | −3.89 |
| 8 | GRK4 | 1.77 | 3.23 | 0.0020 | 0.9832 | −4.06 |
| 9 | FMO5 | 1.59 | 2.68 | 0.0095 | 0.9832 | −4.24 |
| 10 | KCNH3 | −0.65 | −3.49 | 0.0009 | 0.9832 | −4.17 |
| 11 | LRRC46 | −0.12 | 3.26 | 0.0018 | 0.9832 | −4.17 |
| 12 | RNF157 | 1.27 | −2.81 | 0.0066 | 0.9832 | −4.25 |
| 13 | GBGT1 | 3.16 | 2.87 | 0.0056 | 0.9832 | −4.04 |
| 14 | OTOA | −1.99 | 2.98 | 0.0042 | 0.9832 | −4.30 |
| 15 | ANO10 | 5.44 | 3.54 | 0.0008 | 0.9832 | −3.43 |
| 16 | PPIC | 5.54 | 3.36 | 0.0013 | 0.9832 | −3.52 |
| 17 | TM2D2 | 5.54 | 3.23 | 0.0020 | 0.9832 | −3.61 |
| 18 | GPR27 | 2.48 | −3.42 | 0.0011 | 0.9832 | −4.01 |
| 19 | GLDC | 2.06 | −2.92 | 0.0049 | 0.9832 | −4.20 |
| 20 | FAM3B | 0.10 | 2.78 | 0.0072 | 0.9832 | −4.27 |
| 21 | C6orf120 | 4.31 | 3.05 | 0.0034 | 0.9832 | −3.84 |
| 22 | NRG3 | −2.15 | −3.52 | 0.0008 | 0.9832 | −4.23 |
| 23 | KLK12 | −3.18 | 2.74 | 0.0082 | 0.9832 | −4.36 |
| 24 | UTS2B | −2.43 | −3.34 | 0.0014 | 0.9832 | −4.27 |
| 25 | RPS3AP47 | −0.92 | 2.82 | 0.0065 | 0.9832 | −4.30 |
| 26 | IGHV1-3 | −1.24 | −3.10 | 0.0030 | 0.9832 | −4.30 |
| 27 | TAX1BP3 | 5.01 | 3.24 | 0.0019 | 0.9832 | −3.65 |
| 28 | ZSWIM7 | 3.94 | 2.81 | 0.0067 | 0.9832 | −4.00 |
| 29 | ENSG0000 | −3.72 | −3.57 | 0.0007 | 0.9832 | −4.26 |
| | 0218073 | | | | | |
| 30 | FAM228B | 2.52 | 4.17 | 9.84E−05 | 0.6209 | −3.61 |
| 31 | LINC01615 | −0.22 | 2.87 | 0.0056 | 0.9832 | −4.26 |
| 32 | RPS20P14 | −0.05 | 2.84 | 0.0061 | 0.9832 | −4.27 |
| 33 | FAM225B | −0.66 | 2.78 | 0.0072 | 0.9832 | −4.30 |
| 34 | CCT8P1 | −0.13 | 3.95 | 0.0002 | 0.7294 | −3.99 |
| 35 | ENSG0000 | 0.04 | 2.92 | 0.0049 | 0.9832 | −4.24 |
| | 0231747 | | | | | |
| 36 | RPS3AP25 | −3.36 | 3.19 | 0.0023 | 0.9832 | −4.30 |
| 37 | KRT8P39 | −2.74 | −4.18 | 9.38E−05 | 0.6209 | −4.11 |
| 38 | KRT18P5 | −1.92 | −2.94 | 0.0046 | 0.9832 | −4.32 |
| 39 | ENSG0000 | −2.95 | 3.07 | 0.0032 | 0.9832 | −4.31 |
| | 0240211 | | | | | |
| 40 | TCAM1P | −0.02 | −3.55 | 0.0007 | 0.9832 | −4.16 |
| 41 | ENSG0000 | −0.89 | 3.05 | 0.0034 | 0.9832 | −4.25 |
| | 0240401 | | | | | |
| 42 | ENSG0000 | −5.04 | 3.07 | 0.0032 | 0.9832 | −4.35 |
| | 0243635 | | | | | |
| 43 | PPIAP11 | −1.53 | 2.94 | 0.0046 | 0.9832 | −4.30 |
| 44 | LINC01605 | −1.40 | 2.86 | 0.0058 | 0.9832 | −4.30 |
| 45 | ENSG0000 | −2.19 | −3.97 | 0.0002 | 0.7294 | −4.15 |
| | 0255201 | | | | | |
| 46 | ENSG0000 | −0.75 | 3.05 | 0.0034 | 0.9832 | −4.25 |
| | 0257261 | | | | | |
| 47 | ENSG0000 | −3.17 | −3.59 | 0.0007 | 0.9832 | −4.24 |
| | 0258317 | | | | | |
| 48 | ENSG0000 | −2.59 | 3.83 | 0.0003 | 0.9109 | −4.15 |
| | 0261487 | | | | | |
| 49 | ENSG0000 | −2.32 | 2.82 | 0.0064 | 0.9832 | −4.34 |
| | 0261783 | | | | | |
| 50 | ENSG0000 | −0.66 | 2.92 | 0.0049 | 0.9832 | −4.27 |
| | 0261888 | | | | | |
| 51 | ENSG0000 | −2.84 | −3.65 | 0.0005 | 0.9832 | −4.22 |
| | 0262703 | | | | | |
| 52 | ENSG0000 | −2.80 | −2.90 | 0.0051 | 0.9832 | −4.35 |
| | 0263847 | | | | | |
| 53 | ENSG0000 | −3.58 | 3.63 | 0.0006 | 0.9832 | −4.22 |
| | 0267811 | | | | | |
| 54 | ENSG0000 | −3.38 | 2.85 | 0.0059 | 0.9832 | −4.36 |
| | 0269976 | | | | | |
| 55 | ENSG0000 | −1.97 | 3.03 | 0.0036 | 0.9832 | −4.29 |
| | 0271926 | | | | | |
| 56 | ENSG0000 | −3.98 | 2.78 | 0.0073 | 0.9832 | −4.38 |
| | 0272551 | | | | | |
| 57 | ENSG0000 | −3.31 | −2.95 | 0.0045 | 0.9832 | −4.35 |
| | 0275778 | | | | | |
| 58 | ENSG0000 | −2.33 | 2.71 | 0.0086 | 0.9832 | −4.36 |
| | 0280241 | | | | | |

### Example 2—63-Gene Signature Profile in Basal-Like and Luminal Subtype Breast Cancer

Both the basal-like subtype dataset (n=190) and the luminal subtype dataset (n=777) for breast cancer from the TCGA dataset discussed above were analyzed using the 63-gene signature profile.

Overall survival (OS) may be used as a clinical endpoint in trials. OS, while capturing patient deaths due to the studied disease, likewise captures deaths due to other, unrelated causes and is therefore not considered a fully accurate methodology. In addition to or instead of OS, the progression-free interval (PFI), or the period of time during which the cancer does not progress, may also be assessed. Additionally, the disease-free interval (DFI), or the period of time during which a new tumor (either local recurrence or distant metastasis) of the cancer does not develop, was assessed. The minimum follow-up time for PFI is shorter than for OS because patients generally develop disease progression before dying of their disease. PFI, DFI, and OS may be used as endpoints for deriving cancer recurrence signatures.

For the purposes of all of the examples disclosed herein, PFI was scored as a 0 for any patient whose disease did not progress, and a 1 for any patient having a new tumor event, whether it was a progression of disease, local recurrence, distant metastasis, new primary tumors in all sites, or died with the cancer without a new tumor event, including cases with a new tumor event whose type was not available. DFI was scored as a 0 for any patient having no change in disease status, and a 1 for any patient having a new tumor event, whether it was a local recurrence, distant metastasis, or new primary tumor of cancer. OS was scored as a 0 for patients who were still alive, and a 1 for death from any cause. The median follow-up was 2.1 years for all of PFI, DFI, and OS.

Samples were labelled as having a high risk of recurrence or a low risk of recurrence, based upon the recurrence index calculated using gene expression levels of the 63-gene signature, wherein the greater the recurrence index equated to a higher risk of recurrence. In certain analyses, 50% was used as the cutoff for determining high versus low risk. Samples in the top 50^{th} percentile of the recurrence index were labelled as high risk of recurrence, while samples in the bottom 50^{th} percentile of the recurrence index were labelled as low risk of recurrence. In other analyses, 80% was used as the cutoff for determining high versus low risk. Samples in the top 20^{th} percentile of the recurrence index were labelled as high risk of recurrence, while samples in the bottom 80^{th} percentile of the recurrence index were labelled as low risk of recurrence. In yet other analysis, 20% was used as the cutoff for determining high risk versus low risk such that samples in the bottom 20^{th} percentile of the recurrence index were labelled as low risk of recurrence.

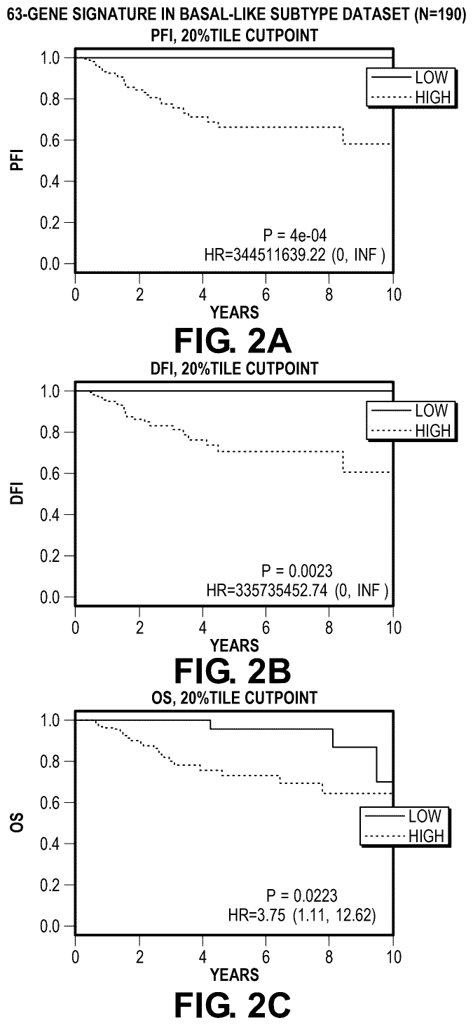

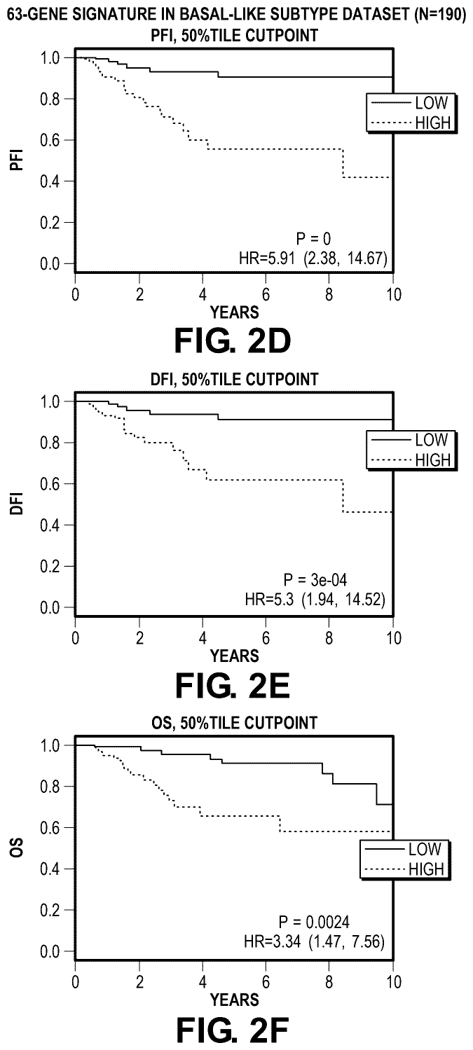

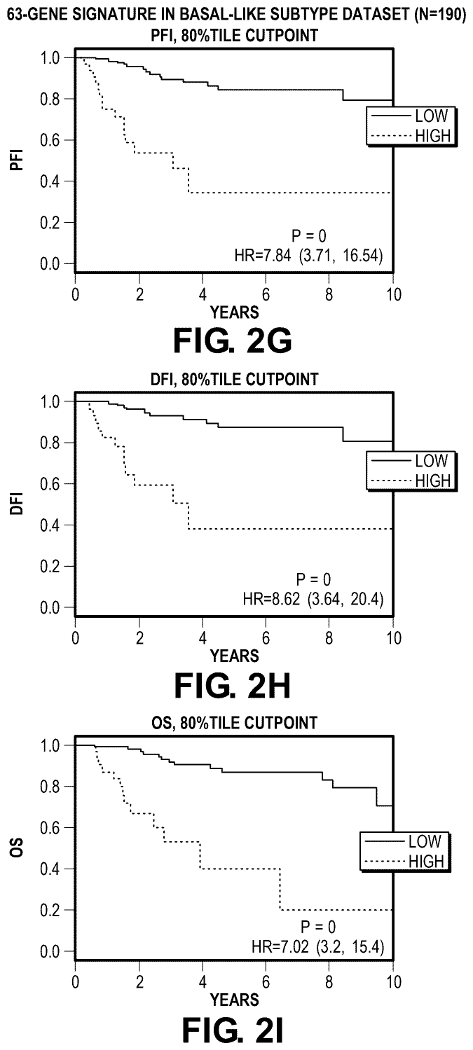

As shown in FIGS. 2A-2C, in the basal-like subtype data set, there was a significant difference between patients identified as having high and low risk of recurrence using a 63-gene signature profile with a 20% cut-off for each of PFI (FIG. 2A), DFI (FIG. 2B), and OS (FIG. 2C). For each of PFI, DFI, and OS, the p-value was 0.0004, 0.0023, and 0.0223, respectively. The hazard ratios for PFI, DFI, and OS were 344511639.22, 335735452.74, and 3.75, respectively. Accordingly, when the 63-gene signature profile was used with a 20% cut-off in the basal-like subtype data set, those classified as high-risk had a statistically significantly higher risk of PFI events than those classified as low-risk, where there were no PFI events recorded in the low-risk group. Likewise, using the secondary endpoint of DFI, the low-risk and high-risk groups were also significantly stratified in the basal-like subtype data set.

As shown in FIGS. 2D-2F, in the basal-like subtype data set, there was a significant difference between patients identified as having high and low risk of recurrence using a 63-gene signature profile with a 50% cut-off for each of PFI (FIG. 2D), DFI (FIG. 2E), and OS (FIG. 2F). For each of PFI, DFI, and OS, the p-value was 0, 0.0003, and 0.0024, respectively, and the hazard ratios for PFI, DFI, and OS were 5.91, 5.3, and 3.34, respectively.

As shown in FIGS. 2G-2I, in the basal-like subtype data set, there was an even greater significant difference between patients identified as having high and low risk of recurrence using a 63-gene signature profile with a 80% cut-off (instead of a 50% cut-off or a 20% cut-off) for each of PFI (FIG. 2G), DFI (FIG. 2H), and OS (FIG. 2I). For each of PFI, DFI, and OS, the p-value was 0, and the hazard ratios for PFI, DFI, and OS were 7.84, 8.62, and 7.02, respectively.

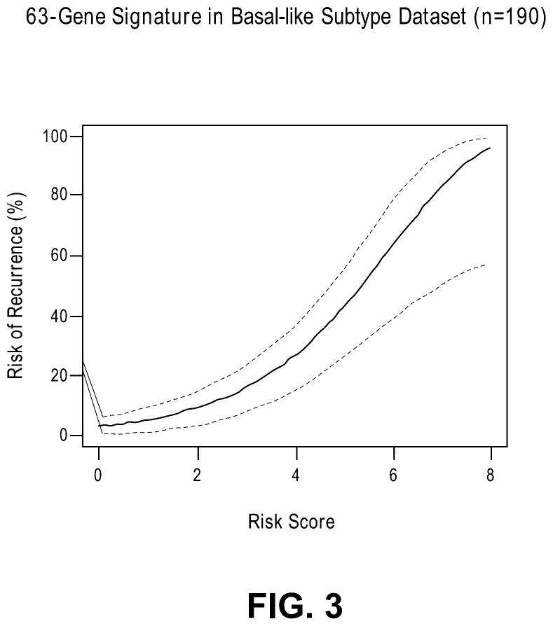

As shown in FIG. 3, for the basal-like subtype group, the 63-gene signature showed an increase risk of recurrence as the recurrence index risk score increased.

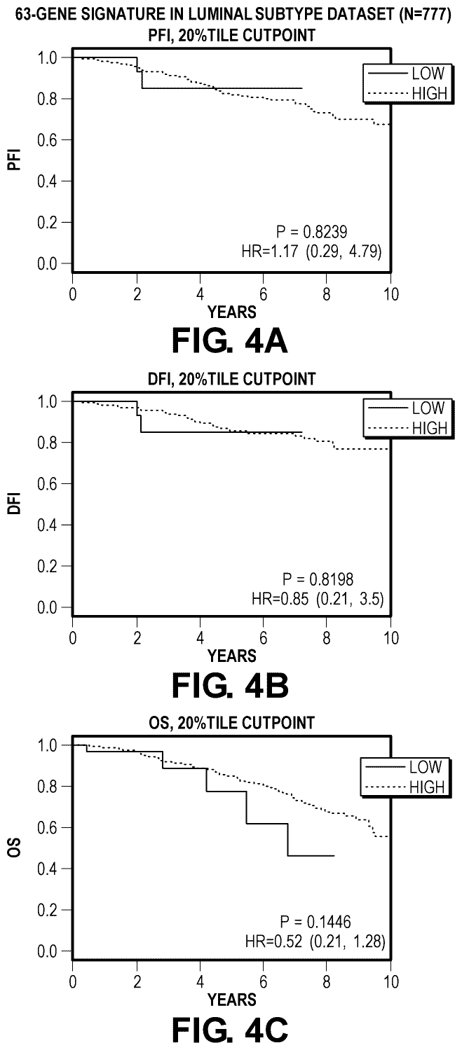

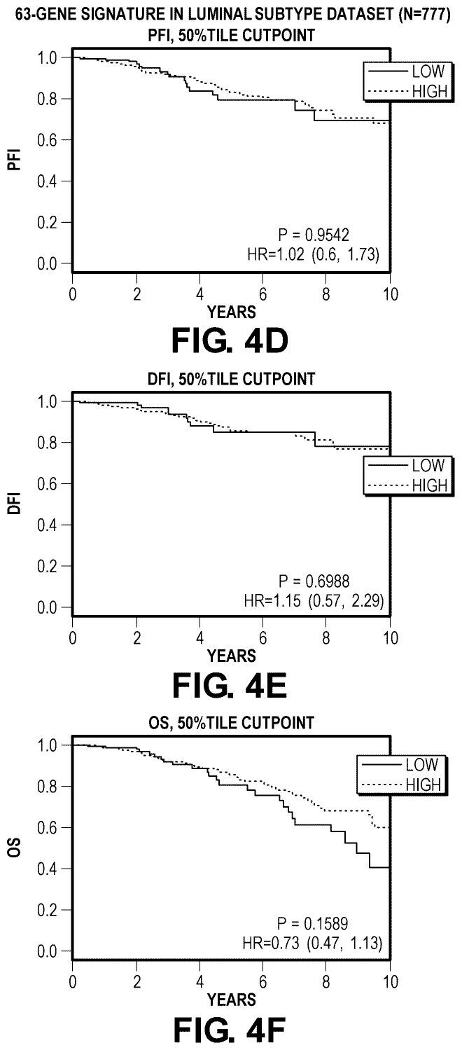

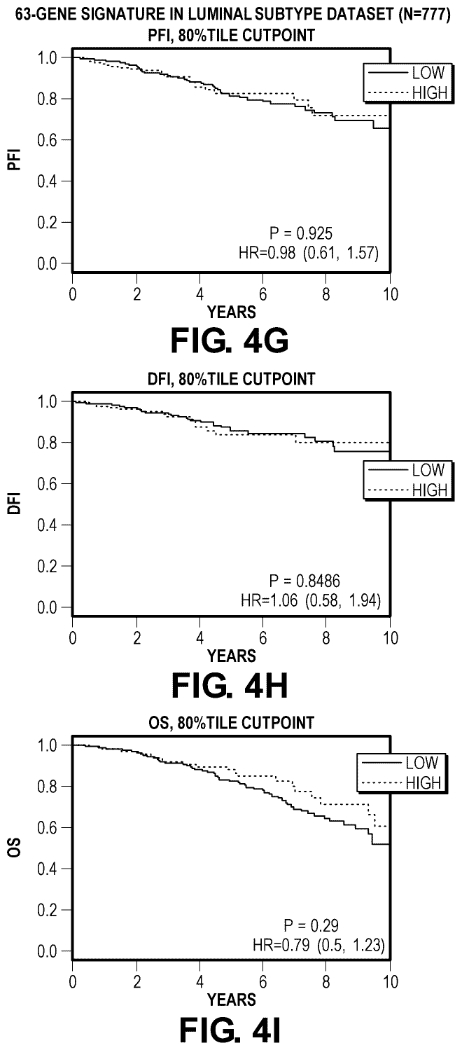

Using the 63-gene signature profile, a significant difference was not observed in the luminal subtype dataset. As shown in FIGS. 4A-4C, in the luminal subtype data set, there was no significant difference between patients identified as having high and low risk of recurrence using a 63-gene signature profile with a 20% cut-off for any of PFI (FIG. 4A), DFI (FIG. 4B), and OS (FIG. 4C). For PFI, DFI, and OS, the p-value was 0.8239, 0.8198, and 0.1446, respectively, and the hazard ratios for PFI, DFI, and OS were 1.17, 0.85, and 0.52, respectively.

As shown in FIGS. 4D-4F, in the luminal subtype data set, there was no significant difference between patients identified as having high and low risk of recurrence using a 63-gene signature profile with a 50% cut-off for any of PFI (FIG. 4D), DFI (FIG. 4E), and OS (FIG. 4F). For PFI, DFI, and OS, the p-value was 0.9542, 0.6988, and 0.1589, respectively, and the hazard ratios for PFI, DFI, and OS were 1.02, 1.15, and 0.73, respectively.

Likewise, as shown in FIGS. 4G-4I, in the luminal subtype data set, there was no significant difference between patients identified as having high and low risk of recurrence using a 63-gene signature profile with a 80% cut-off (instead of a 50% cut-off) for any of PFI (FIG. 4G), DFI (FIG. 4H), and OS (FIG. 4I). For PFI, DFI, and OS, the p-value was 0.98, 0.8486, and 0.29, respectively, and the hazard ratios for PFI, DFI, and OS were 0.98, 1.06, and 0.79, respectively.

### Example 3—63-Gene Signature in High-Grade Serous Ovarian Cancer

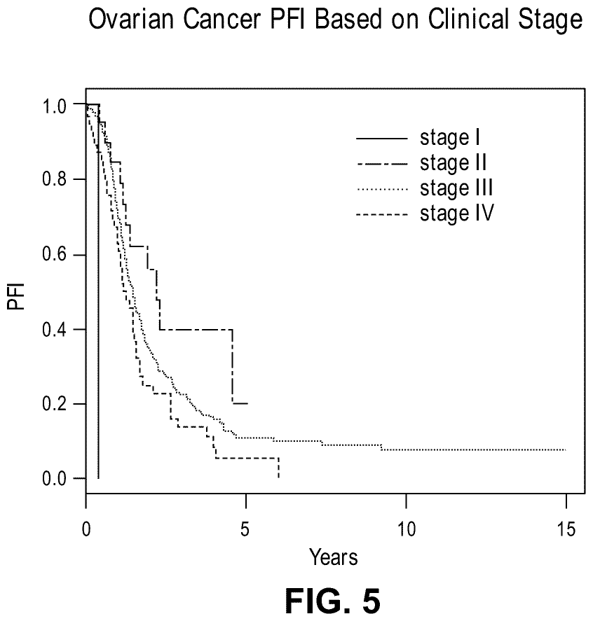

The 63-gene signature was used to evaluate a patient's chance for high or low risk of PFI, DFI, and OS after a high-grade serous ovarian cancer diagnosis. The high-grade serous ovarian cancer patient samples were categorized based on the stage of high-grade serous ovarian cancer, i.e., Stage I, II, III, and IV. Table 7A below details the patients' clinical characteristics from the TCGA data set. As shown in Table 7A, 93% of the patients were diagnosed as Stage III or IV, and 86% were Grade 3. FIG. 5 shows a Kaplan-Meier plot of the PFI for the high-grade serous ovarian cancer patients (n=371) by Stage I, II, III, and IV. As expected, patients diagnosed as Stage III or IV have a poor prognosis. Accordingly, the 80^{th} percentile was chosen as the cut-off point for determining high risk of recurrence.

TABLE 7A
Stage I-IV high-grade serous ovarian cancer
patient clinical characteristics

| | | RNA-seq (N = 371) | |
|---|---|---|---|
| Factors | | N (%) | |
| Age | Median (min, max) | 59 | (30, 87) |
| Race | White | 324 | (87%) |
| | Black | 25 | (7%) |
| | Asian | 11 | (3%) |
| | Unknown | 14 | (3%) |
| Clinical Stage | I | 1 | (0%) |
| | II | 21 | (6%) |
| | III | 292 | (78%) |
| | IV | 57 | (15%) |
| | Unknown | 3 | (1%) |
| Grade | G1 | 1 | (0%) |
| | G2 | 42 | (11%) |
| | G3 | 320 | (86%) |
| | G4 | 1 | (0%) |
| | GX | 10 | (3%) |
| Overall Survival | Death | 230 | (61%) |
| | Alive | 144 | (39%) |
| Disease-Free | Event | 126 | (34%) |
| Interval (DFI) | Event-free | 51 | (13%) |
| | Unknown | 197 | (53%) |
| Progression-Free | Event | 272 | (73%) |
| Interval (PFI) | Event-free | 102 | (27%) |

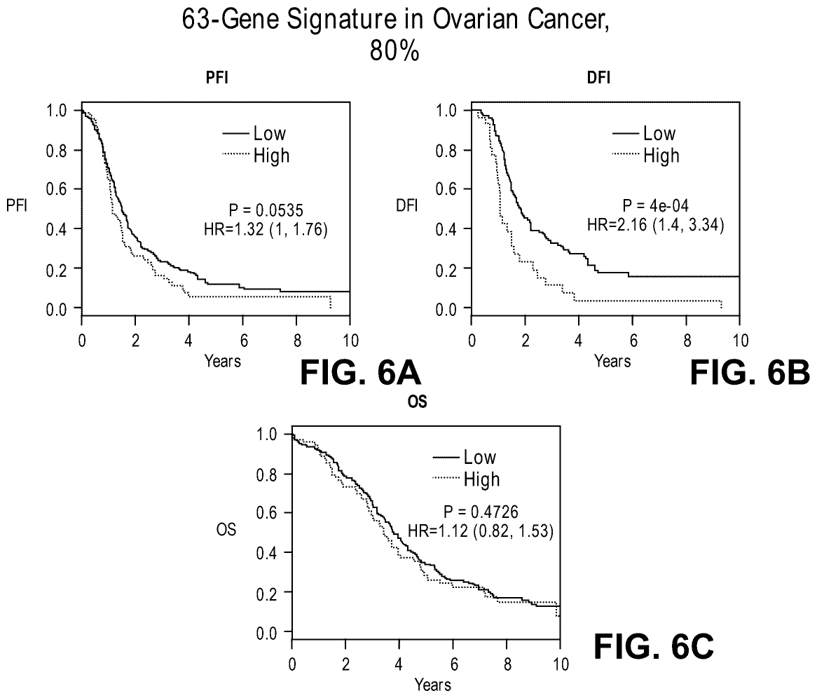

Using the 63-gene profile, a slight difference was noted between PFI and DFI, but not OS. As shown in FIGS. 6A, across the entire high-grade serous ovarian cancer data set (n=374), there was a difference indicating a strong trend, albeit not significant, for PFI (p-value=0.0535), for high and low risk of recurrence when the 63-gene signature profile was used with an 80% cut-off; the hazard ratio for PFI was 1.32. As shown in FIG. 6B, there was a significant difference for DFI (p-value=0.0004), for high and low risk of recurrence when the 63-gene signature profile was used with an 80% cut-off, and the hazard ratio was 2.16. As shown in FIG. 6C, there was no significant difference for OS (p-value=0.4726), for high and low risk of recurrence when the 63-gene signature profile was used with an 80% cut-off, and the hazard ratio was 1.12.

The dataset was next analyzed in the absence of the Stage IV and unknown stage patients, using only patients diagnosed as Stage I, II, and III. Table 7B below details the clinical data for the 314 samples used in the analyses that follow.

TABLE 7B
Stage I-III high-grade serous ovarian
cancer patient clinical characteristics

| | | RNA-seq (N = 314) | |
|---|---|---|---|
| Factors | | N (%) | |
| Age | Median (min, max) | 59 | (30, 87) |
| Race | White | 269 | (86%) |
| | Black | 23 | (7%) |
| | Asian | 9 | (3%) |
| | Unknown | 13 | (4%) |
| Clinical Stage | I | 1 | (0%) |
| | II | 21 | (7%) |
| | III | 292 | (93%) |
| Grade | G2 | 35 | (11%) |
| | G3 | 273 | (87%) |
| | GX | 6 | (2%) |
| Disease-Free | Event | 126 | (40%) |
| Interval (DFI) | Event-free | 50 | (16%) |

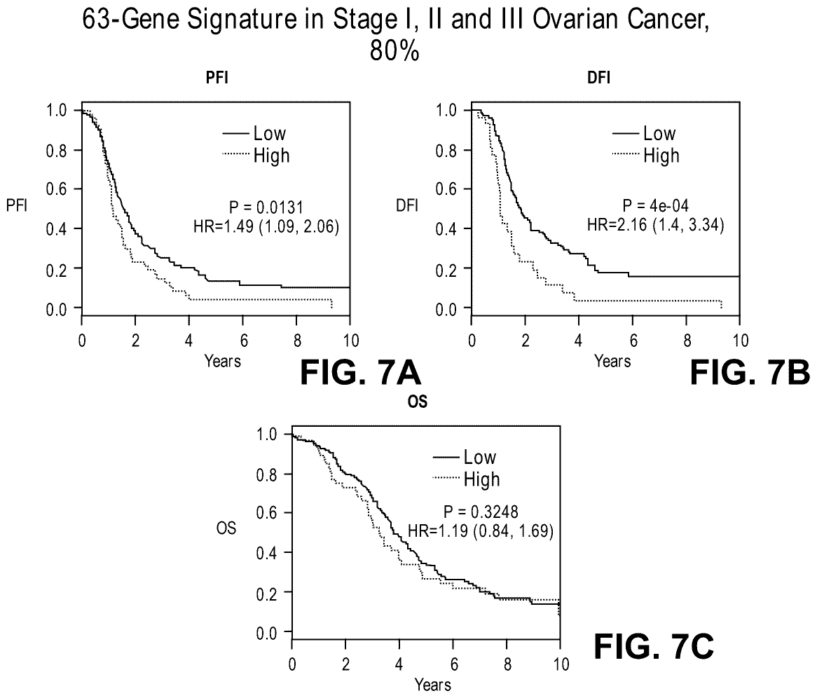

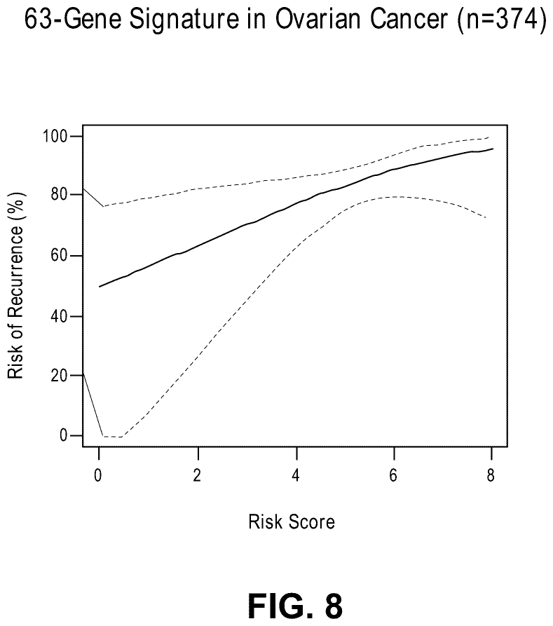

As shown in FIGS. 7A-7C, there was a significant difference between patients identified as having high and low risk of recurrence using a 63-gene signature profile with an 80% cut-off for both PFI and DFI; there was not, however, a significant difference in OS over a 10 year period. As shown in FIGS. 7A and 7B, PFI and DFI were significantly different (p-value=0.0131 and p-value=0.0004, respectively), and the hazard ratios for PFI and DFI were 1.49 and 2.16, respectively. For OS, the p-value was 0.3248 with a hazard ratio of 1.19, as shown in FIG. 7C. As shown in FIG. 8, for the high-grade serous ovarian cancer patient group, the 63-gene signature showed an increase risk of recurrence as the recurrence index risk score increased.

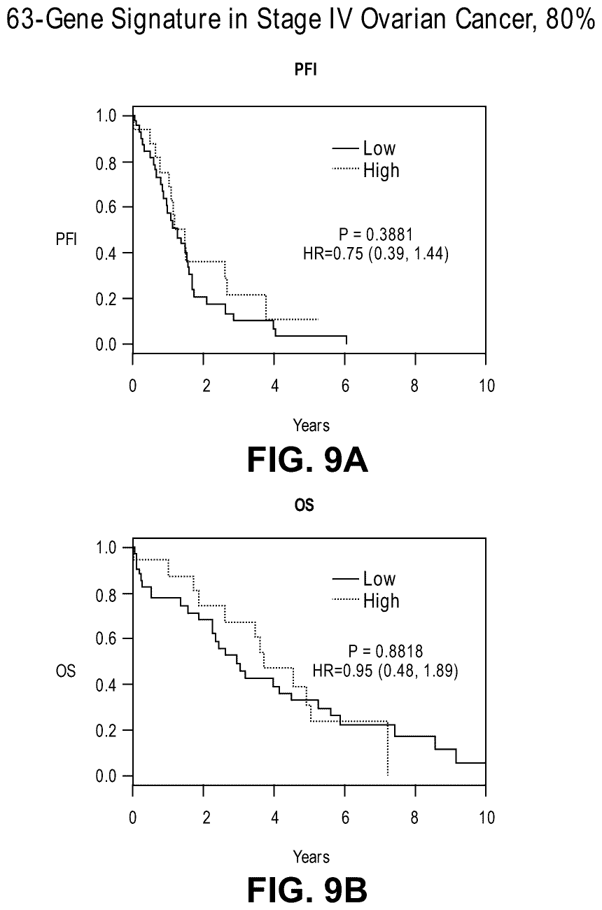

When analyzing the dataset for only the Stage IV patients, there was, as expected, no significant difference between either PFI (p-value=0.3881) or OS (p-value=0.8818). See FIGS. 9A and 9B. The hazard ratios for PFI and OS were 0.75 and 0.95, respectively.

### Example 4—58-Gene Signature in Basal-Like and Luminal Subtype Breast Cancer

Both the basal-like subtype dataset (n=190) and the luminal subtype dataset (n=777) for breast cancer from the TCGA dataset discussed above were analyzed using the 58-gene signature profile. As discussed above, PFI, DFI, and OS were scored either as “1” or “0.”

As in Example 2, samples were labelled as having a high risk of recurrence or a low risk of recurrence, based upon a recurrence index calculated using the gene expression levels of the 58-gene signature, wherein the greater the recurrence index equated to a higher risk of recurrence. Analyses were conducted using both a 50% cutoff and an 80% cutoff to determine whether samples were designated either as having a high or low risk of recurrence.

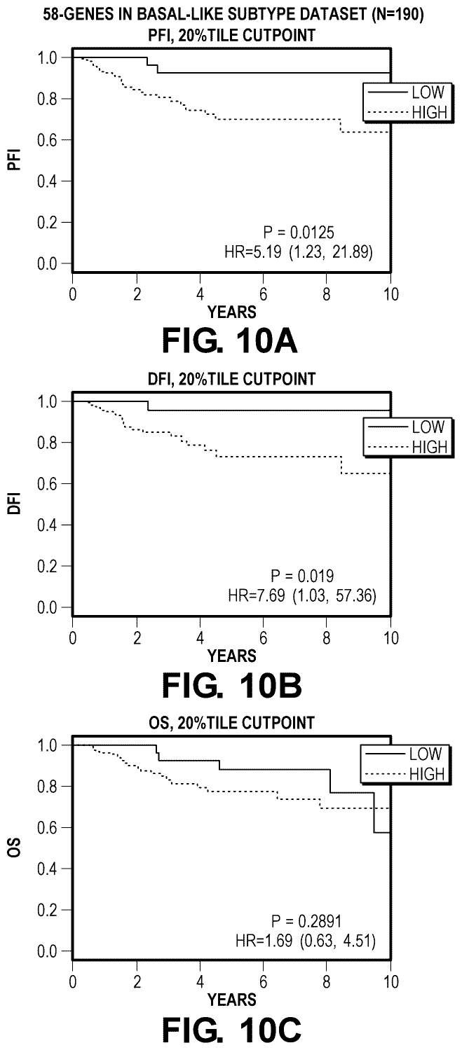

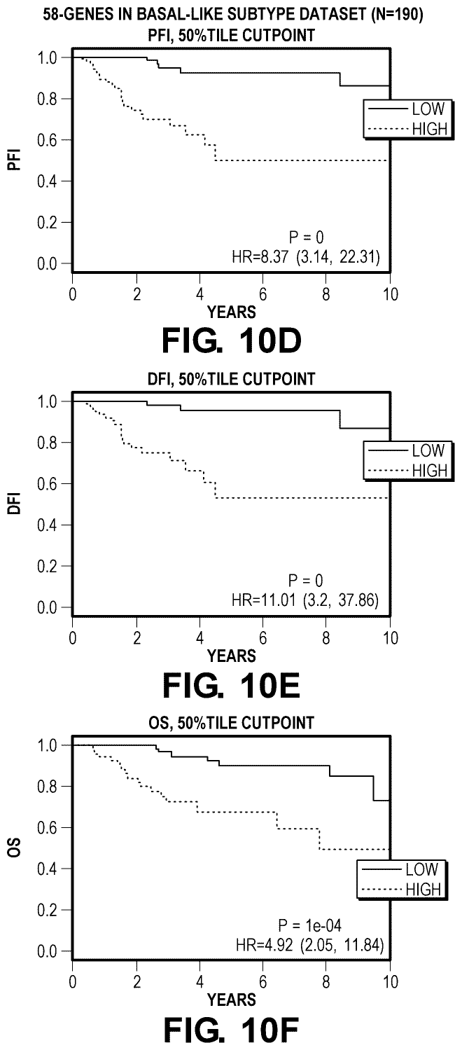

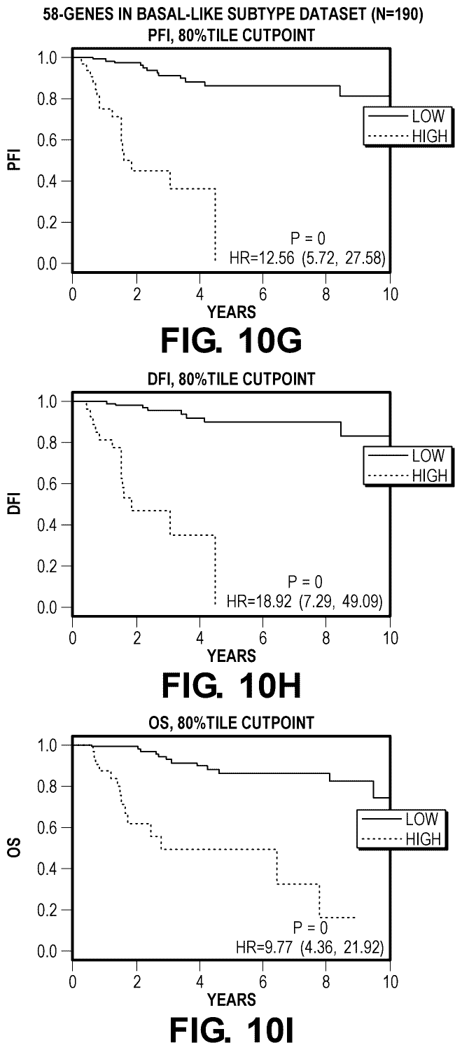

As shown in FIGS. 10A-10C, in the basal-like subtype data set, there was a significant difference between patients identified as having high and low risk of recurrence using a 58-gene signature profile with a 20% cut-off for both PFI (FIG. 10A) and DFI (FIG. 10B), although the difference was not significant for OS (FIG. 10C). For PFI, DFI, and OS, the p-value was 0.0125, 0.019, and 0.2891, respectively, and the hazard ratios for PFI, DFI, and OS were 5.19, 1.03, and 1.69, respectively.

As shown in FIGS. 10D-10F, in the basal-like subtype data set, there was a significant difference between patients identified as having high and low risk of recurrence using a 58-gene signature profile with a 50% cut-off for each of PFI (FIG. 10D), DFI (FIG. 10E), and OS (FIG. 10F). For each of PFI, DFI, and OS, the p-value was 0, 0, and 0.0001, respectively, and the hazard ratios for PFI, DFI, and OS were 8.37, 11.01, and 4.92, respectively.

As shown in FIGS. 10G-10H, in the basal-like subtype data set, there was an even greater significant difference between patients identified as having high and low risk of recurrence using a 58-gene signature profile with a 80% cut-off (instead of a 50% cut-off) for each of PFI (FIG. 10G), DFI (FIG. 10H), and OS (FIG. 10I). For all of PFI, DFI, and OS, the p-value was 0, and the hazard ratios for PFI, DFI, and OS were 12.56, 18.92, and 9.77, respectively.

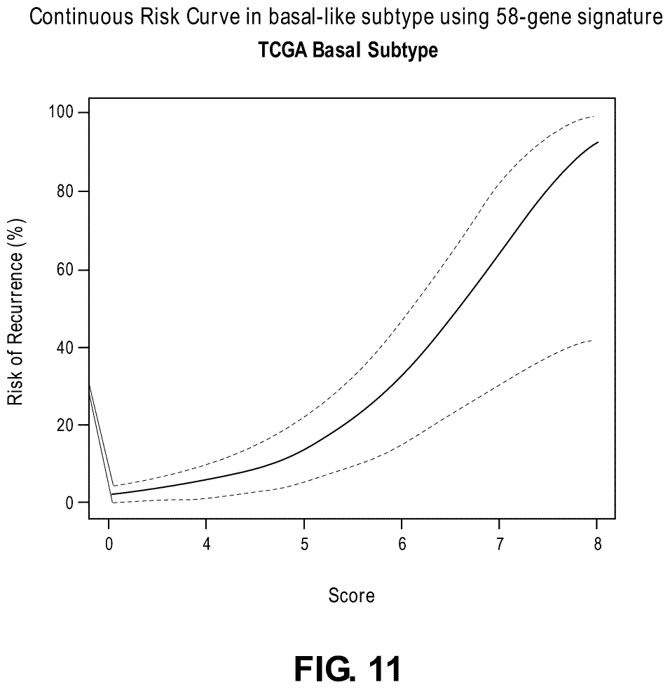

As shown in FIG. 11, for the basal-like subtype group, the 58-gene signature showed an increase risk of recurrence as the recurrence index risk score increase.

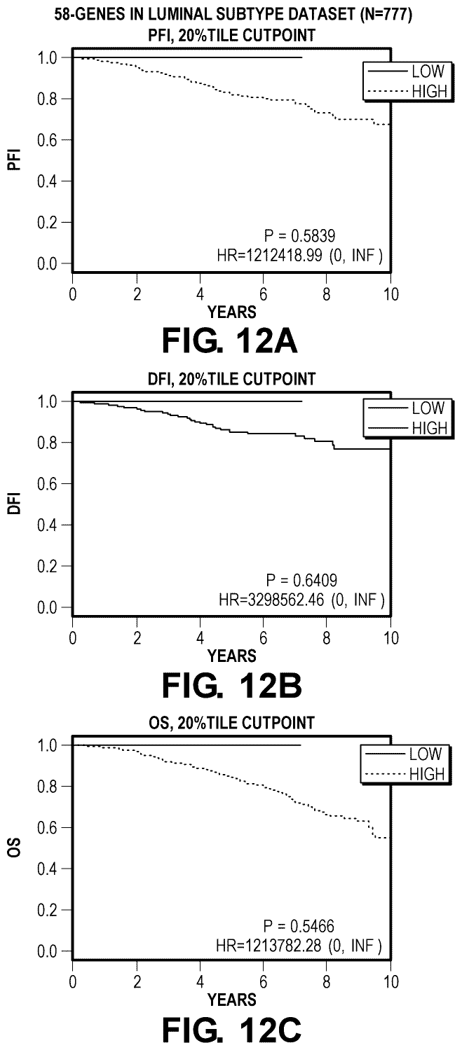

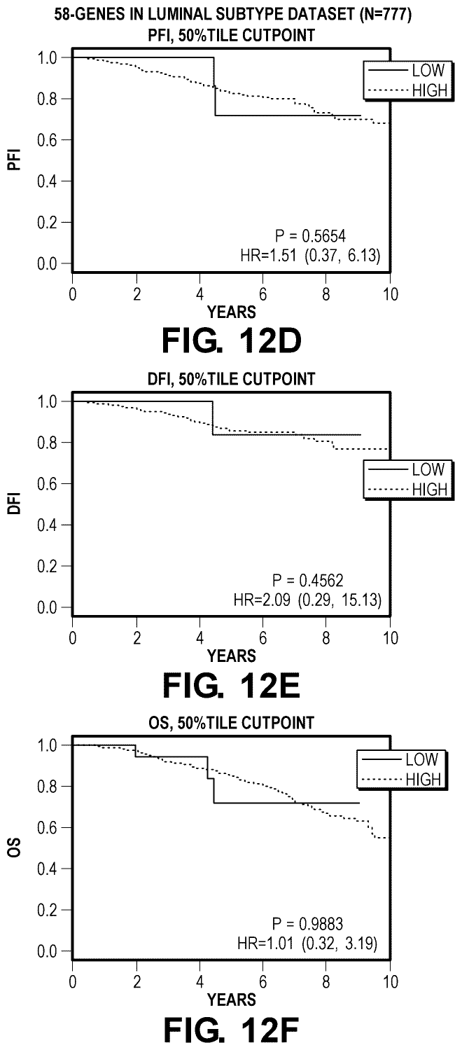

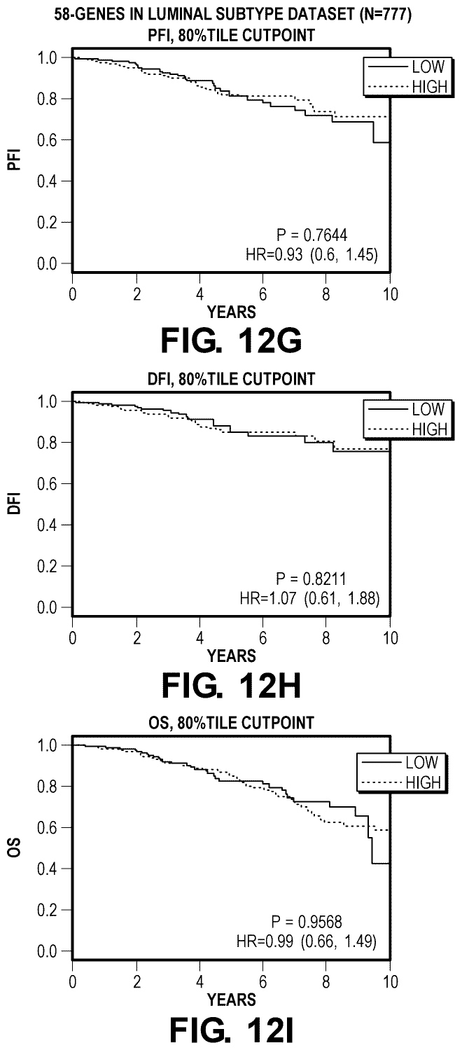

Using the 58-gene signature profile, a significant difference was not observed in the luminal subtype dataset. As shown in FIGS. 12A-12C, in the luminal subtype data set, there was no significant difference between patients identified as having high and low risk of recurrence using a 58-gene signature profile with a 20% cut-off for any of PFI (FIG. 12A), DFI (FIG. 12B), and OS (FIG. 12C). For PFI, DFI, and OS, the p-value was 0.5839, 0.6409, and 0.5466, respectively, and the hazard ratios PFI, DFI, and OS were 1212418.99, 3298562.46, and 1213782.28, respectively.

As shown in FIGS. 12D-12F, in the luminal subtype data set, there was no significant difference between patients identified as having high and low risk of recurrence using a 58-gene signature profile with a 50% cut-off for any of PFI (FIG. 12D), DFI (FIG. 12E), and OS (FIG. 12F). For PFI, DFI, and OS, the p-value was 0.5654, 0.4562, and 0.9883, respectively, and the hazard ratios PFI, DFI, and OS were 1.51, 2.09, and 1.01, respectively.

Likewise, as shown in FIGS. 12G-12I, in the luminal subtype data set, there was no significant difference between patients identified as having high and low risk of recurrence using a 58-gene signature profile with a 80% cut-off (instead of a 50% cut-off) for any of PFI (FIG. 12G), DFI (FIG. 12H), and OS (FIG. 12I). For PFI, DFI, and OS, the p-value was 0.7644, 0.8211, and 0.9568, respectively, and the hazard ratios for PFI, DFI, and OS were 0.93, 1.07, and 0.99, respectively.

### Example 5—58-Gene Signature in High-Grade Serous Ovarian Cancer

The 58-gene signature was used to evaluate a patient's chance for high or low risk of PFI, DFI, and OS after a high-grade serous ovarian cancer diagnosis. Data were derived from the TCGA dataset as shown in Table 7A above. As in Example 3, the 80^{th} percentile was chosen as the cut-off point for determining high risk of recurrence, given the poor prognosis of the patients in the dataset.

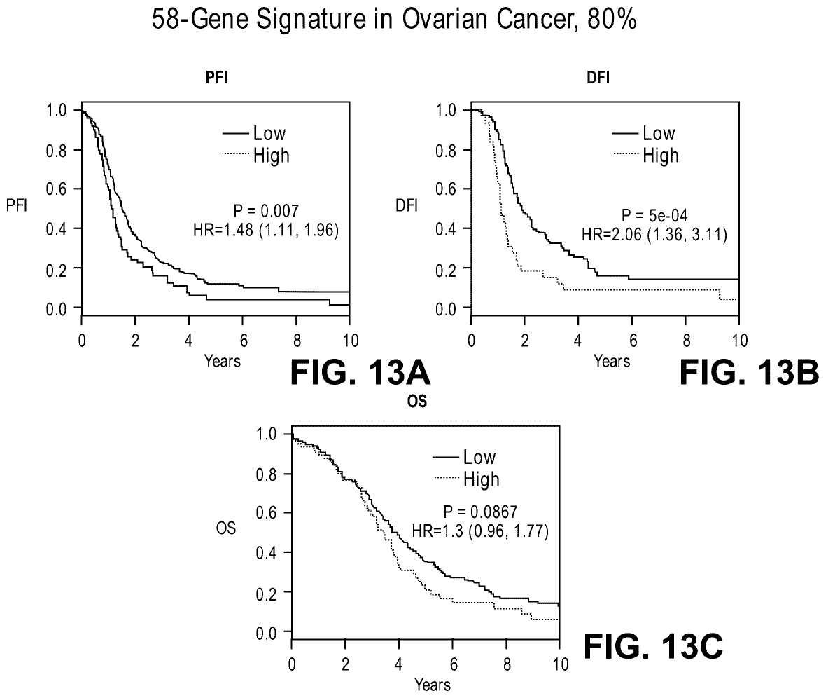

Using the 58-gene profile, a significant difference was noted between PFI and DFI, but not OS. As shown in FIGS. 13A, across the entire high-grade serous ovarian cancer data set (n=374), a significant difference for PFI (p-value=0.007) was observed, for high and low risk of recurrence when the 58-gene signature profile was used with an 80% cut-off; the hazard ratio for PFI was 1.48. As shown in FIG. 13B, there was also significant difference for DFI (p-value=0.0005), for high and low risk of recurrence when the 58-gene signature profile was used with an 80% cut-off, and the hazard ratio was 2.06. As shown in FIG. 13C, there was no significant difference for OS (p-value=0.0867), for high and low risk of recurrence when the 58-gene signature profile was used with an 80% cut-off, and the hazard ratio was 1.3.

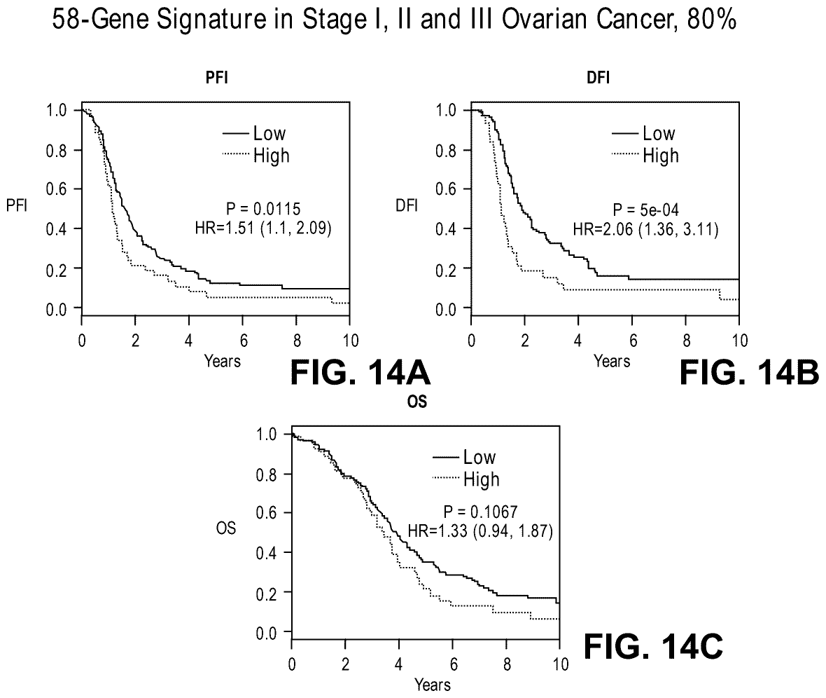

The dataset was next analyzed in the absence of the Stage IV and unknown stage patients, using only patients diagnosed as Stage I, II, and III. As shown in FIGS. 14A-14C, there was a significant difference between patients identified as having high and low risk of recurrence using a 58-gene signature profile with a 80% cut-off for both PFI and DFI; there was not, however, a significant difference in OS over a 10 year period. As shown in FIGS. 14A and 14B, PFI and DFI were significantly different (p-value=0.0115 and p-value=0.0005, respectively), and the hazard ratios for PFI and DFI were 1.51 and 2.06, respectively. For OS, the p-value was 0.1067 with a hazard ratio of 1.33, as shown in FIG. 14C.

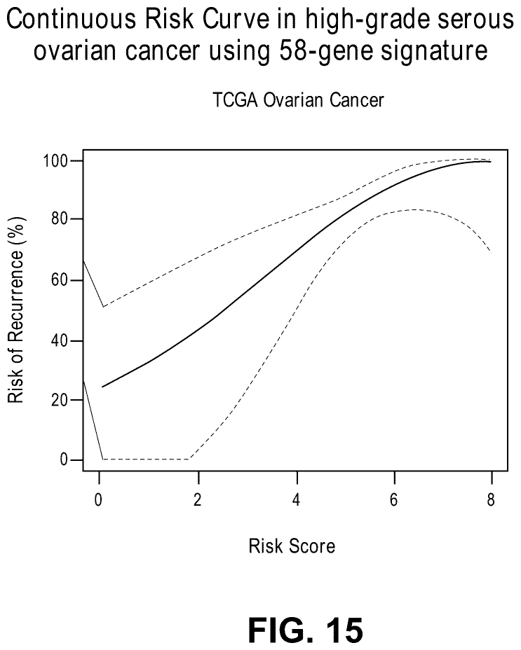

As shown in FIG. 15, for the high-grade serous ovarian cancer patient group, the 58-gene signature showed an increase risk of recurrence as the recurrence index risk score increased.

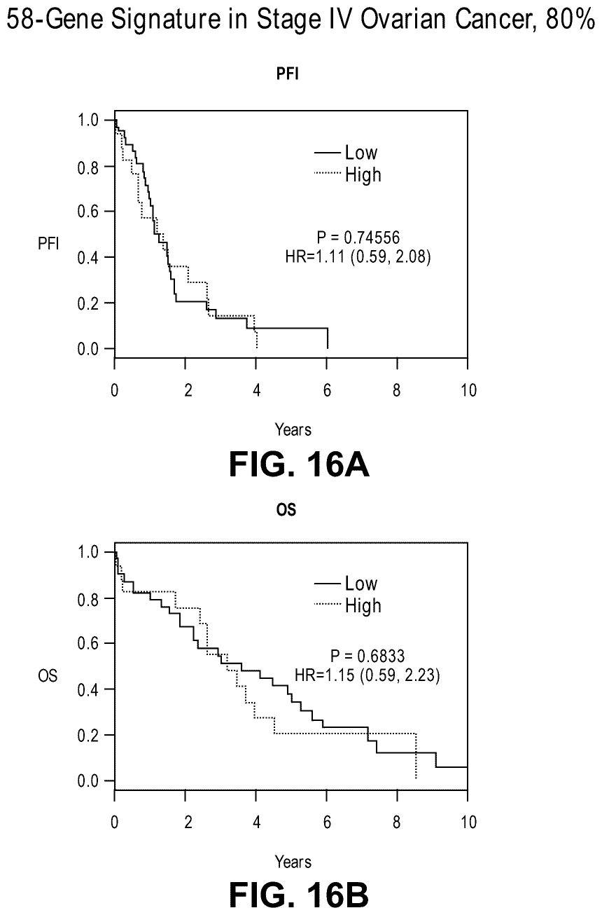

When analyzing the dataset for only the Stage IV patients, there was, as expected, no significant difference between either PFI (p-value=0.74556) or OS (p-value=0.6813). See FIGS. 16A and 16B. The hazard ratios for PFI and OS were 1.11 and 1.15, respectively.

### Example 6—Gene Ontology Term Enrichment Analysis for 63-Gene Signature

The Gene Ontology (GO) database is the world's largest source of information on the function of genes and provides a foundation for computational analysis of large-scale molecular biology and genetics experiments in biomedical research. To further explore and validate the 63-gene signature identified herein, GO enrichment analysis was performed on the gene signature.

Given a set of 43 genes (excluding 10 RNA genes and 10 unmapped genes), enrichment analysis was performed from the geneontology.org webpage. The gene list was entered into the GO Enrichment Analysis box powered by the PANTHER classification system and “biological processes” and “Homo sapiens” were selected for the domain and species, respectively.

The resulting enrichment analysis indicated 156 gene ontology (GO) terms that were over-represented (p<0.05). No GO terms were significant after adjustment of the false discovery rate (FDR), but the results nonetheless are indicative of biological meaning.

18 GO terms had a p-value of less than 0.01. Among them was the vascular endothelial growth factor (VEGF) signaling pathway. Research has previously linked VEGF signaling to cancer. See, e.g., Inai, T. et al, Inhibition of vascular endothelial growth factor (VEGF) signaling in cancer causes loss of endothelial fenestrations, regression of tumor vessels, and appearance of basement membrane ghosts, AM J PATHOL. 2004; 165(1):35-52 and Kowanetz, M. & Ferrara, N., Vascular Endothelial Growth Factor Signaling Pathways: Therapeutic Perspective, CLIN CANCER RES 2006; 12(17):5018-22 (showing that VEGF is released by tumor cells and induces tumor neovascularization, which represents a target for antitumor therapy).

A second GO term that was identified is “cell-cell signaling,” which regulates cell proliferation, motility, and survival. A third GO term was “peptide hormone processing,” which involves control of the biology of individual cells, organs, and organisms. In tumor cells, these peptide hormone processes may result in uncontrolled growth as a consequence of autocrine and/or paracrine growth effects. Treston, A. M. et al., Control of tumor cell biology through regulation of peptide hormone processing, J NATL CANCER INST MONOGR 1992; 13:169-75. The other 18 GO terms include metabolic processes, such asphthalate metabolic process and phytoalexin metabolic process, which affect the metabolic processes of a tumor. See, e.g., Hsieh T. H. et al., Phthalates induce proliferation and invasiveness of estrogen receptor-negative breast cancer through the AhR/HDAC6/c-Myc signaling pathway, FASEB J. 2012; 26(2):778-87.

Several of the GO terms having a p-value between 0.01 and 0.05 were also indicative of a biological meaning. For instance, for “CD8 positive T-cell differentiation,” it is well-known that tumor-infiltrating T-cells may play a role in tumor progression. Furthermore, cell cycle progression may affect integrin expression and DNA repair mechanisms, and changes in cellular metabolism are associated with the activation of diverse immune subsets. Kedia-Mehta N, et al., Competition for nutrients and its role in controlling immune responses. Nature Communications, NATURE COMM 2019; 10:2123.

The results from the GO enrichment analysis demonstrate the association between the recurrence 63-gene signature and cancer biological process, further validate its biological meaning, and support its utility for clinical application and target drug therapy.

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

1. A method of obtaining a gene expression profile in a biological sample from a patient, the method comprising:
detecting expression of a plurality of genes in a biological sample obtained from the patient, wherein the plurality of genes comprises at least 5 of the following 63 human genes: PTHLH, LAMB4, P2RX6, OLFM4, CLEC11A, SLC5A5, HSPB1, RPA3, PRMT8, PCDHB5, TRIM67, PGF, PAX1, KLHDC7B, DISP2, LRRC46, P3H4, TM4SF19, SCUBE1, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, IGHV1-3, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, TUNAR, LINC01605, BLOC1S5-TXNDC5, ENSG00000261409, ENSG00000261487, ENSG00000261888, YTHDF3-AS1, ENSG00000271959, ENSG00000272551, ENSG00000272732, and ENSG00000281383.

2. (canceled)

3. The method of claim 1, wherein the plurality of genes comprises at least the following human genes:
RPA3, LRRC46, ANO10, LINC01605, FAM3B, FAM228B, IGHV1-3, ENSG00000231747, ENSG00000240401, ENSG00000261487, ENSG00000261888, and ENSG00000272551.

4-5. (canceled)

6. A method of predicting cancer recurrence in a patient, comprising:
determining the expression levels of a plurality of genes in a biological sample obtained from the patient, wherein the plurality of genes comprises at least 5 of the following 63 human genes: PTHLH, LAMB4, P2RX6, OLFM4, CLEC11A, SLC5A5, HSPB1, RPA3, PRMT8, PCDHB5, TRIM67, PGF, PAX1, KLHDC7B, DISP2, LRRC46, P3H4, TM4SF19, SCUBE1, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, IGHV1-3, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, TUNAR, LINC01605, BLOC1S5-TXNDC5, ENSG00000261409, ENSG00000261487, ENSG00000261888, YTHDF3-AS1, ENSG00000271959, ENSG00000272551, ENSG00000272732, and ENSG00000281383;
determining differential gene expression based on reduced or enhanced expression levels of the plurality of genes compared to a control non-recurrent cancer sample;
calculating a recurrence index for the patient based on the gene expression levels; and
identifying the patient as having a high risk of cancer recurrence if the recurrence index is above a threshold.

7-13. (canceled)

14. The method of claim 1, wherein nucleic acid expression is detected.

15. The method of claim 1, wherein polypeptide expression is detected.

16-19. (canceled)

20. A kit for use in predicting cancer recurrence and/or prognosing cancer, the kit comprising a plurality of probes for detecting at least 5 of the following 63 human genes:
PTHLH, LAMB4, P2RX6, OLFM4, CLEC11A, SLC5A5, HSPB1, RPA3, PRMT8, PCDHB5, TRIM67, PGF, PAX1, KLHDC7B, DISP2, LRRC46, P3H4, TM4SF19, SCUBE1, ANO10, VPS28, SCGB3A1, MT2P1, LINC01116, CA3, OPRPN, CSN3, KCNK3, GLIS1, TVP23C, PCSK1, SRRM3, EXOSC4, TH, ZNF703, FAM3B, KLK12, MUC12, IGHV1-3, ENSG00000213757, FAM228B, LINC01615, RPS20P14, ENSG00000225840, TEX41, DNM3OS, LINC00704, ENSG00000231747, ENSG00000240401, VSIG8, LINC02432, ENSG00000249780, TUNAR, LINC01605, BLOC1S5-TXNDC5, ENSG00000261409, ENSG00000261487, ENSG00000261888, YTHDF3-AS1, ENSG00000271959, ENSG00000272551, ENSG00000272732, and ENSG00000281383,
wherein the plurality of probes contains probes for detecting no more than 500 different genes.

21-29. (canceled)

30. The method of claim 1, wherein the plurality of genes comprises at least 15 of the following human genes:
PTHLH, RPA3, PRMT8, PCDHB5, PAX1, KLHDC7B, LRRC46, TM4SF19, SCUBE1, ANO10, CA3, CSN3, KCNK3, GLIS1, TVP23C, ZNF703, FAM3B, MUC12, IGHV1-3, FAM228B, LINC00704, ENSG00000231747, ENSG00000240401, VSIG8, LINC02432, TUNAR, LINC01605, BLOC1S5-TXNDC5, ENSG00000261487, ENSG00000261888, and ENSG00000272551.

31. The method of claim 1, wherein the plurality of genes comprises at least 20 of the following human genes:
PTHLH, RPA3, PRMT8, PCDHB5, PAX1, KLHDC7B, LRRC46, TM4SF19, SCUBE1, ANO10, CA3, CSN3, KCNK3, GLIS1, TVP23C, ZNF703, FAM3B, MUC12, IGHV1-3, FAM228B, LINC00704, ENSG00000231747, ENSG00000240401, VSIG8, LINC02432, TUNAR, LINC01605, BLOC1S5-TXNDC5, ENSG00000261487, ENSG00000261888, and ENSG00000272551.

32. The method of claim 1, wherein the plurality of genes comprises at least 30 of the following human genes:
PTHLH, RPA3, PRMT8, PCDHB5, PAX1, KLHDC7B, LRRC46, TM4SF19, SCUBE1, ANO10, CA3, CSN3, KCNK3, GLIS1, TVP23C, ZNF703, FAM3B, MUC12, IGHV1-3, FAM228B, LINC00704, ENSG00000231747, ENSG00000240401, VSIG8, LINC02432, TUNAR, LINC01605, BLOC1S5-TXNDC5, ENSG00000261487, ENSG00000261888, and ENSG00000272551.

33. The method of claim 1, wherein the plurality of genes comprises the following human genes:
PTHLH, RPA3, PRMT8, PCDHB5, PAX1, KLHDC7B, LRRC46, TM4SF19, SCUBE1, ANO10, CA3, CSN3, KCNK3, GLIS1, TVP23C, ZNF703, FAM3B, MUC12, IGHV1-3, FAM228B, LINC00704, ENSG00000231747, ENSG00000240401, VSIG8, LINC02432, TUNAR, LINC01605, BLOC1S5-TXNDC5, ENSG00000261487, ENSG00000261888, and ENSG00000272551.

34. The method of claim 32, wherein an array is used to detect expression of the plurality of genes and wherein the array has polynucleotide probes for no more than 250 genes.

35. The method of claim 32, wherein an array is used to detect expression of the plurality of genes and wherein the array has polynucleotide probes for no more than 50 genes.

36. The method of claim 32, wherein an array is used to detect expression of the plurality of genes and wherein the array has polynucleotide probes for no more than 40 genes.

37. The method of claim 3, wherein an array is used to detect expression of the plurality of genes and wherein the array has polynucleotide probes for no more than 50 genes.

38. The method of claim 32, wherein an array is used to detect expression of the plurality of genes and wherein the array has no more than 250 different addressable elements.

39. The method of claim 32, wherein an array is used to detect expression of the plurality of genes and wherein the array has fewer than 50 different addressable elements.

40. The method of claim 32, wherein an array is used to detect expression of the plurality of genes and wherein the array has fewer than 40 different addressable elements.

41. The method of claim 3, wherein an array is used to detect expression of the plurality of genes and wherein the array has fewer than 50 different addressable elements.
